(19) United States
(12) Patent Application Publication
(10) Pub. No.: US 2011/0059172 A1
(43) Pub. Date: Mar. 10, 2011

(54) THERAPEUTIC COMPOSITIONS

(76) Inventors: Arthur Tzianabos, Reading, MA (US); Michael W. Heartlein, Boxborough, MA (US)

(21) Appl. No.: 12/865,094

(22) PCT Filed: Jan. 28, 2009
(86) PCT No.: PCT/US09/32263

§ 371 Date: Nov. 17, 2010

Related U.S. Application Data
(60) Provisional application No. 61/024,451, filed on Jan. 29, 2008.

(51) Int. Cl. A61K 38/43; A61K 38/16; A61K 38/47; A61K 38/46; A61K 9/00; A61P 1/16; A61P 25/00; A61P 21/00; A61P 19/00; A61K 9/14

(52) U.S. Cl. 424/484; 514/1.1; 424/94.5; 424/94.61; 424/94.6; 427/2.14

(57) ABSTRACT

Therapeutic compositions containing therapeutic agents and poly(beta-amino esters) or polymers thereof are described. These tertiary amine-containing polymers are preferably biodegradable and biocompatible. Nanoparticles and microparticles containing polymer/therapeutic agent complexes are also described.

### CROSS REFERENCE TO RELATED APPLICATIONS

This application claims priority to U.S. Application Ser. No. 61/024,451, filed on Jan. 29, 2008. The disclosure of the prior application is considered part of (and is incorporated by reference in) the disclosure of this application.

### BACKGROUND

Deficient or low levels of a protein can lead to a disease or disorder in an individual. The protein at issue can be therapeutically administered to the individual with deficient or low levels of the protein to restore or increase the levels of the protein in the individual, for example, as a method of treating a disease or disorder caused by, or associated with, the deficient or low protein levels.

### SUMMARY

Polymers (e.g., polymers containing poly(beta-amino esters)) can be used in the preparation of therapeutic compositions that contain a therapeutic protein, e.g., a protein that can be used in replacement therapy. As used herein, “replacement therapy” refers to the use of a protein to reconstitute a deficiency or to increase otherwise low levels of the protein, e.g., in an individual that has a disease or disorder (or has a predisposition for a disease or disorder) caused by, or associated with, a protein deficiency or by low levels of a protein. The therapeutic composition can be contained in a pharmaceutical composition.

The protein deficiency or low levels may be caused, for example, by mutation (e.g., in a gene encoding the protein or in an element controlling expression of the gene (e.g., a regulatory sequence)), misfolding of the protein, or truncation of the protein (e.g., an amino or carboxy terminal truncation). The protein may be an enzyme. Non-limiting examples of proteins (e.g., therapeutic proteins) that can be used in replacement therapy include Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, and N-acetylgalactosamine-4-sulfatase.

In one aspect, the invention features a therapeutic composition comprising a polymer described herein (e.g., a polymer containing poly(beta-amino esters)) and a therapeutic agent, wherein the therapeutic agent comprises a protein, e.g., a therapeutic protein, e.g., a protein for use in replacement therapy, e.g., a protein described herein.

In some aspects, the disclosure features a therapeutic composition that contains a therapeutic agent (e.g., a therapeutic protein, e.g., a protein for use in replacement therapy, e.g., a protein described herein) and a compound of the formula:

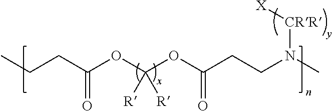

wherein

X is methyl, OR or NR2;

R is selected from the group consisting of hydrogen, alkyl, alkenyl, alkynyl, cyclic, heterocyclic, aryl, and heteroaryl; each R′ is independently selected from the group consisting of hydrogen, C1-C6 lower alkyl, C1-C6 lower alkoxy, hydroxy, amino, alkylamino, dialkylamino, cyano, thiol, heteroaryl, aryl, phenyl, heterocyclic, carbocyclic, and halogen; n is an integer between 3 and 10,000; x is an integer between 1 and 10; y is an integer between 1 and 10; and derivatives and salts thereof.

In some embodiments, the therapeutic agent is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In some embodiments, the compound is amine-terminated.

In some embodiments, the compound is acrylate-terminated.

In some embodiments, x is an integer between 2 and 7, e.g., x is 4; e.g., x is 5; e.g., x is 6.

In some embodiments, y is an integer between 2 and 7, e.g., y is 4; e.g., y is 5; e.g., y is 6.

In some embodiments, wherein R is hydrogen. In some preferred embodiments, R is methyl, ethyl, propyl, butyl, pentyl, or hexyl.

In some embodiments, the structure of the compound is

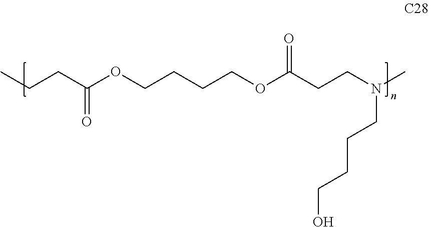

and R is hydrogen, x is 4, and y is 4.

In some embodiments, the structure of the compound is

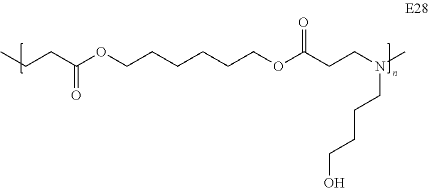

and R is hydrogen, x is 6, and y is 4.

In some embodiments, the structure of the compound is

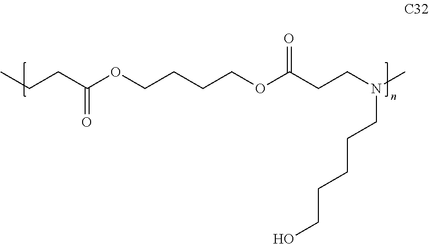

and R is hydrogen, x is 4, and y is 5.

In some embodiments, the structure of the compound is

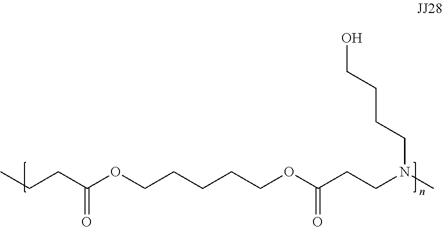

and R is hydrogen, x is 5, and y is 4.

In some embodiments, the structure of the compound is

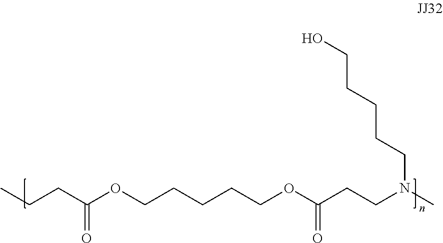

and R is hydrogen, x is 5, and y is 5.

In some embodiments, the structure of the compound is

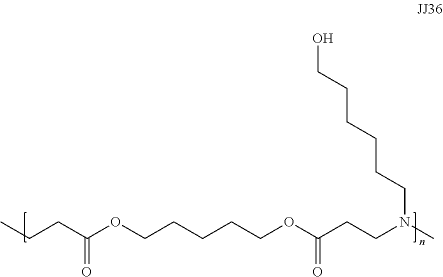

and R is hydrogen, x is 5, and y is 6.

In some aspects, the disclosure features a therapeutic composition that includes a therapeutic agent (e.g., a therapeutic protein, e.g., a protein for use in replacement therapy, e.g., a protein described herein) and a compound of the formula:

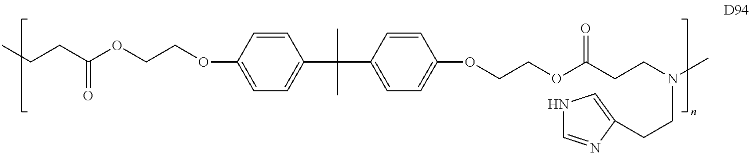

wherein n is an integer between 3 and 10,000; and derivatives and salts thereof.

In some embodiments, the therapeutic agent is a therapeutic protein that includes Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In other aspects, the disclosure features a therapeutic composition that contains a therapeutic agent (e.g., a therapeutic protein, e.g., a protein for use in replacement therapy, e.g., a protein described herein) and a compound of the formula:

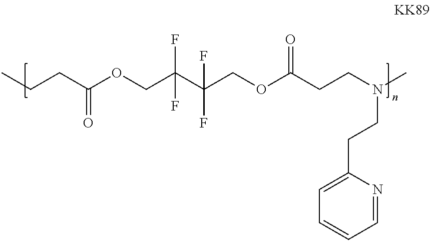

wherein n is an integer between 3 and 10,000; and derivatives and salts thereof.

In some embodiments, the therapeutic agent is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In other aspects, the disclosure features a therapeutic composition that includes a therapeutic agent (e.g., a therapeutic protein, e.g., a protein for use in replacement therapy, e.g., a protein described herein) and a compound of the formula:

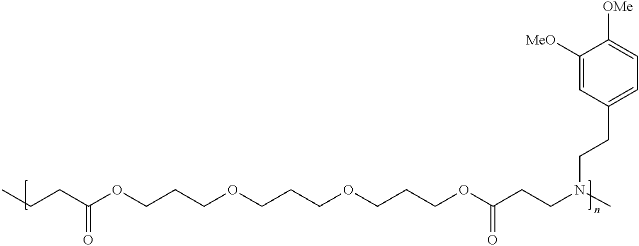

(M17) wherein n is an integer between 3 and 10,000; and derivatives and salts thereof.

In some embodiments, the therapeutic agent is a therapeutic protein that includes Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In some aspects, the disclosure features a therapeutic composition that contains a therapeutic agent (e.g., a therapeutic protein, e.g., a protein for use in replacement therapy, e.g., a protein described herein) and a compound of the formula:

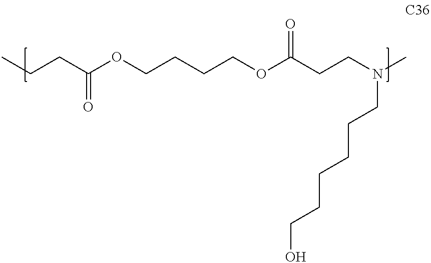

wherein n is an integer between 3 and 10,1000; and derivatives and salts thereof.

In some embodiments, the therapeutic agent is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In other aspects, the disclosure features a therapeutic composition that contains a therapeutic agent (e.g., a therapeutic protein, e.g., a protein for use in replacement therapy, e.g., a protein described herein) and polymer selected from the group consisting of: C86, D60, B14, G5, D61, U94, F32, F28, JJ36, JJ32, LL6, LL8, U28, E28, U36, E36, U32, E32, C94, F94, JJ94, U28, JJ86, C86, U86, E86, C80, E80, JJ80, U80, D24, E24, JJ24, B17, II28, II36, II32, C20, JJ20, E20, C25, U25, D25, D70, D28, D32, D36, D93, U87, D87, C75, U75,020, 028, C94, AA20, AA28, D86, F86, AA36, AA24, AA94,024, AA60, A61, C32, JJ28, C28, JJ20, D94, U32, D24, C36, E28, D36, U94, E24, E32, D28, U36, E80, E36, JJ80, E94, D93, B17, M17, AA61, U93, and C25; wherein the acrylates A-PP are of the formula:

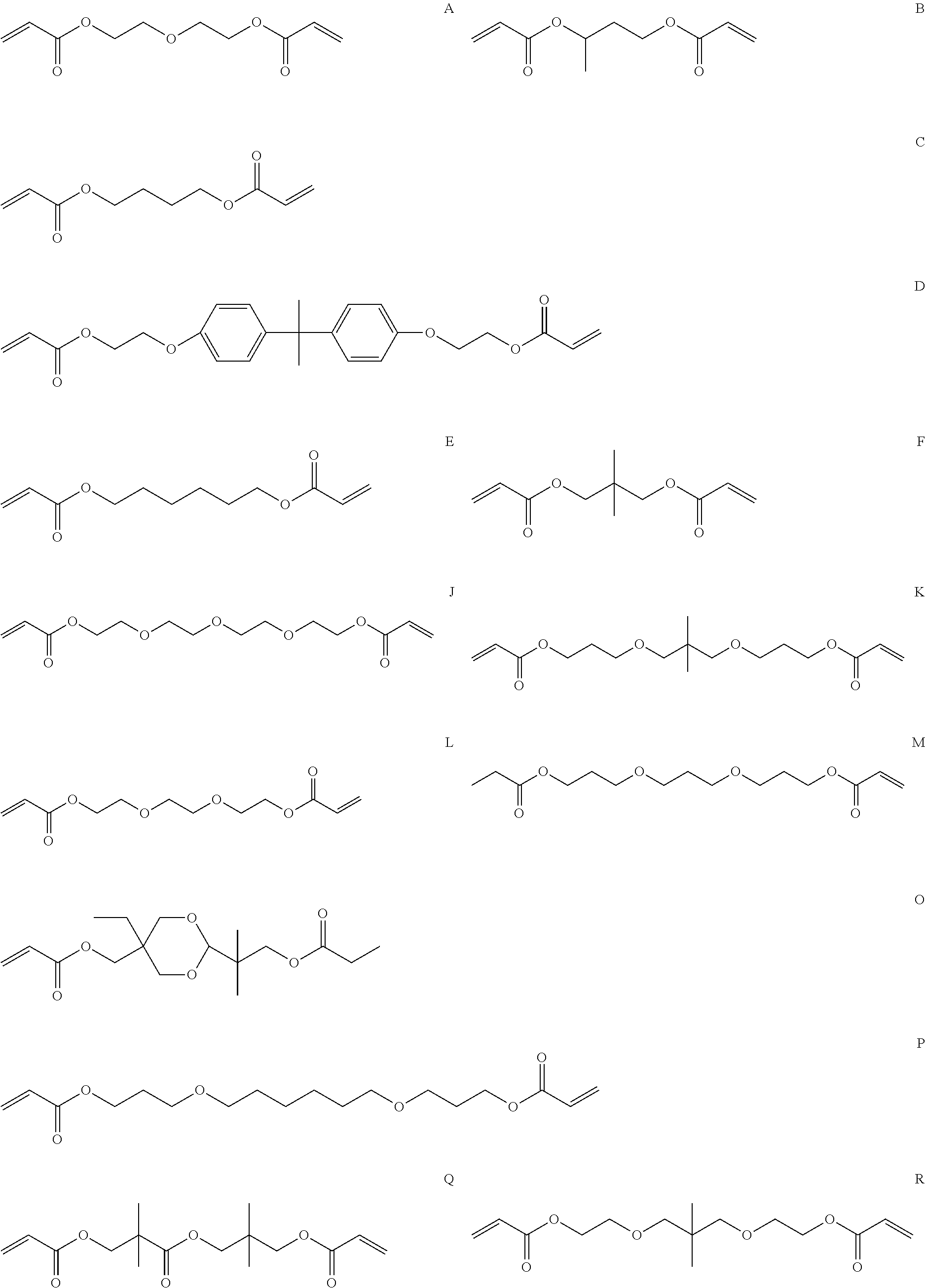

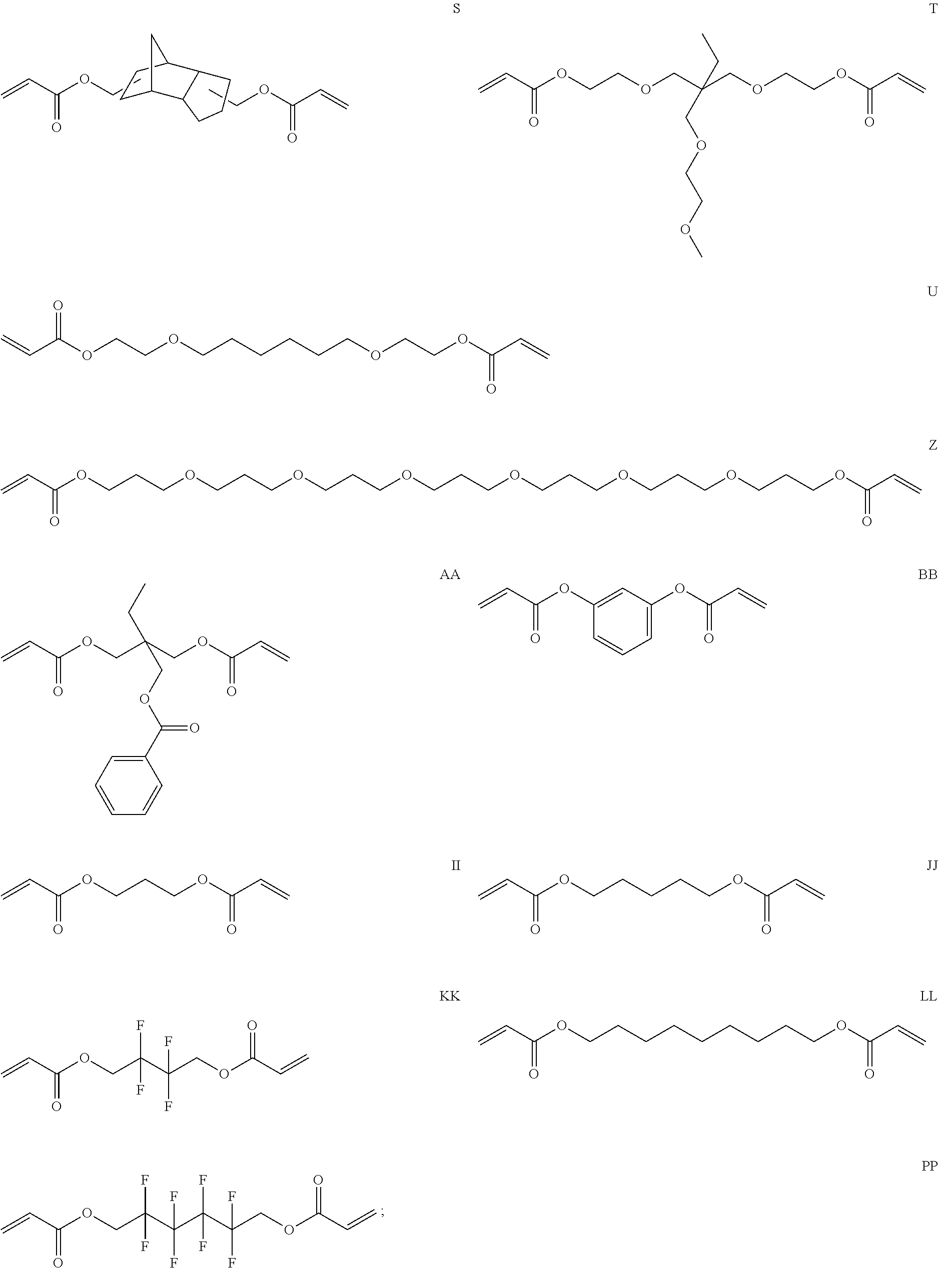

and the amines 1-94 are of the formula:

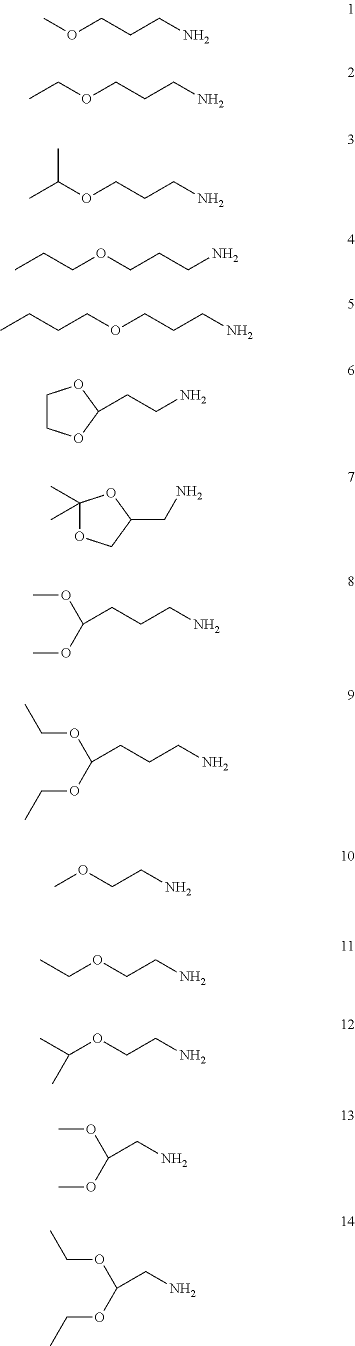

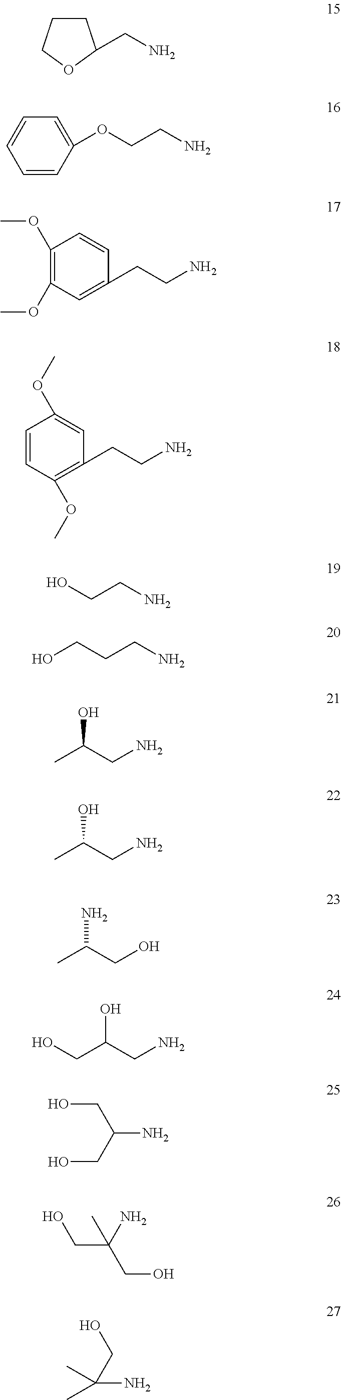

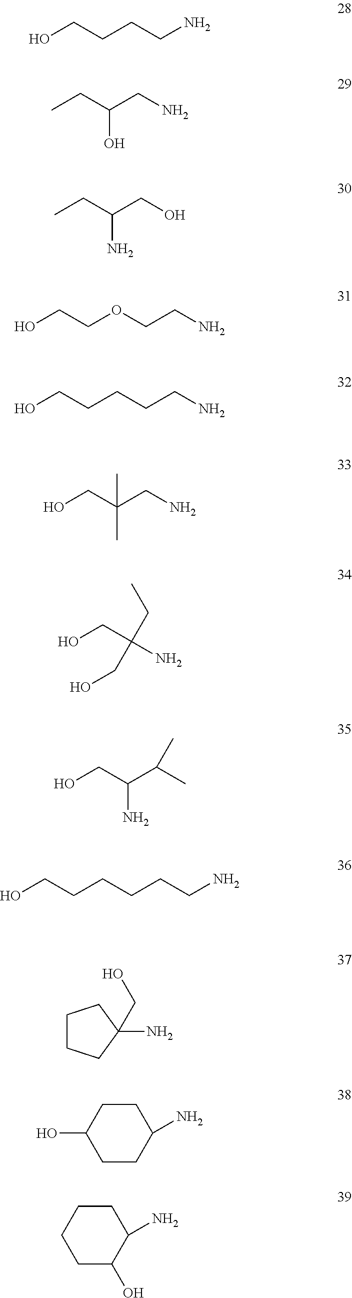

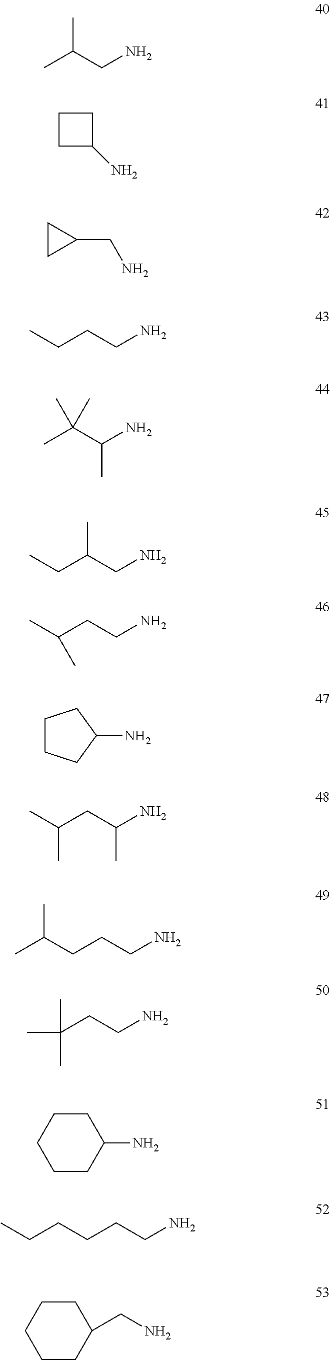

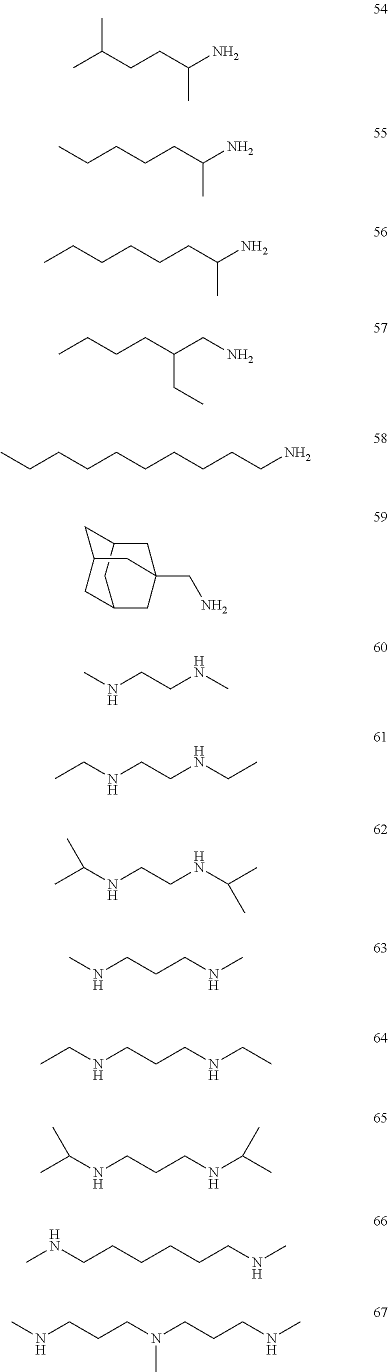

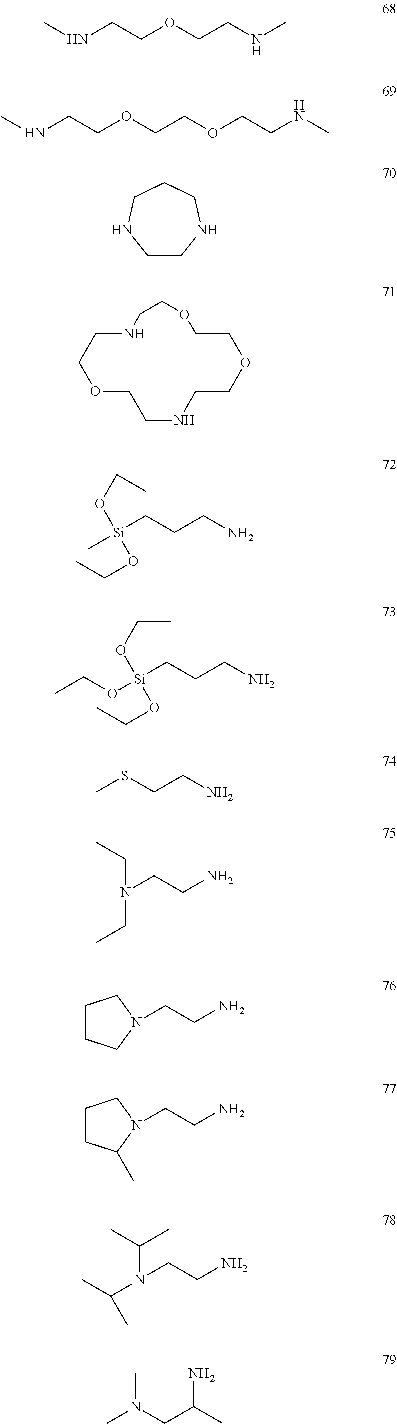

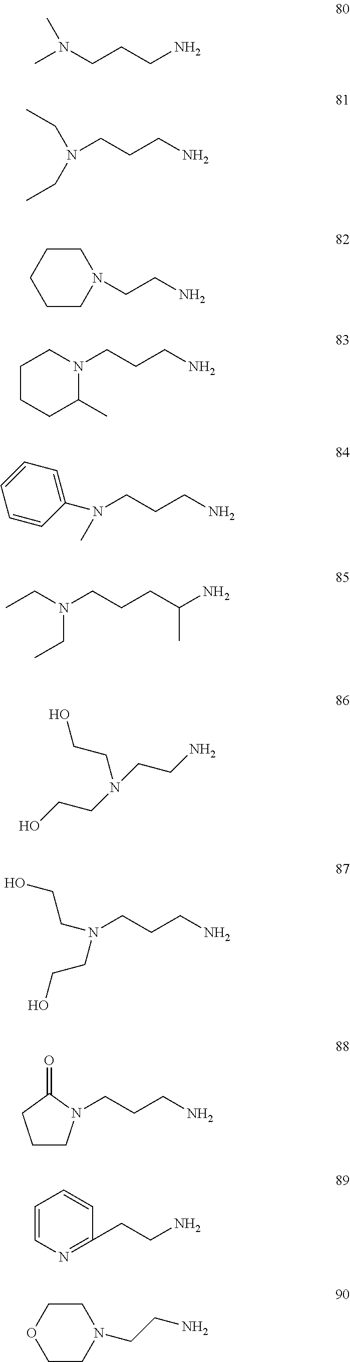

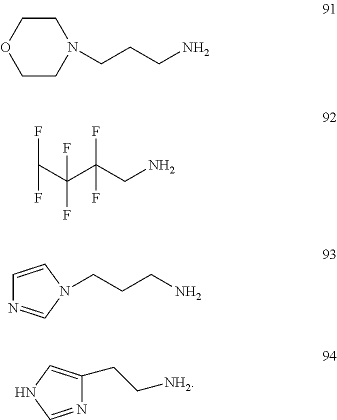

In some embodiments, the therapeutic agent is a therapeutic protein that includes Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In other aspects, the disclosure features a therapeutic composition that includes a therapeutic agent (e.g., a therapeutic protein, e.g., a protein for use in replacement therapy, e.g., a protein described herein) and a poly(beta-amino ester) comprising a bis(acrylate ester) selected from the group consisting of formulae A-PP:

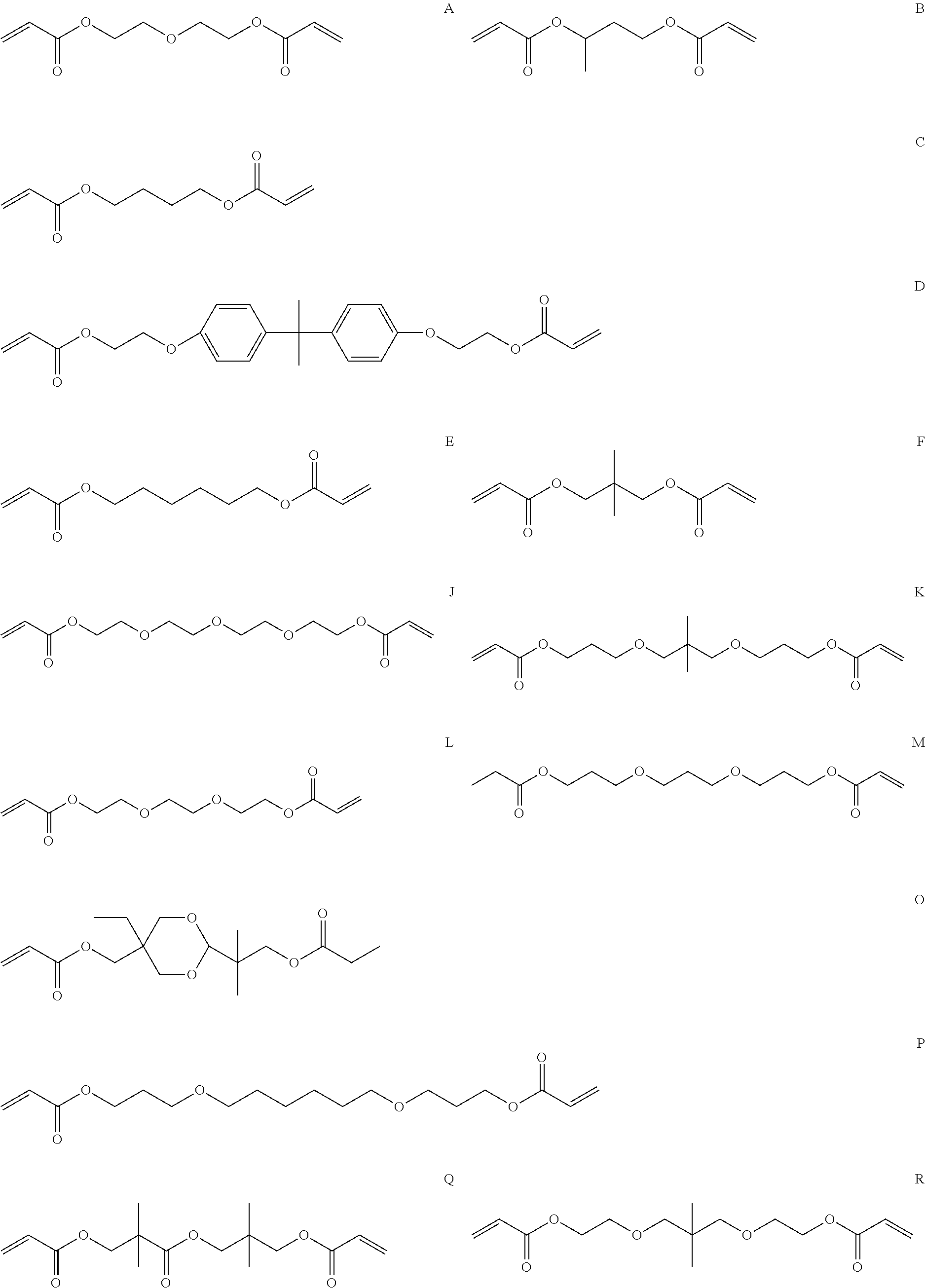

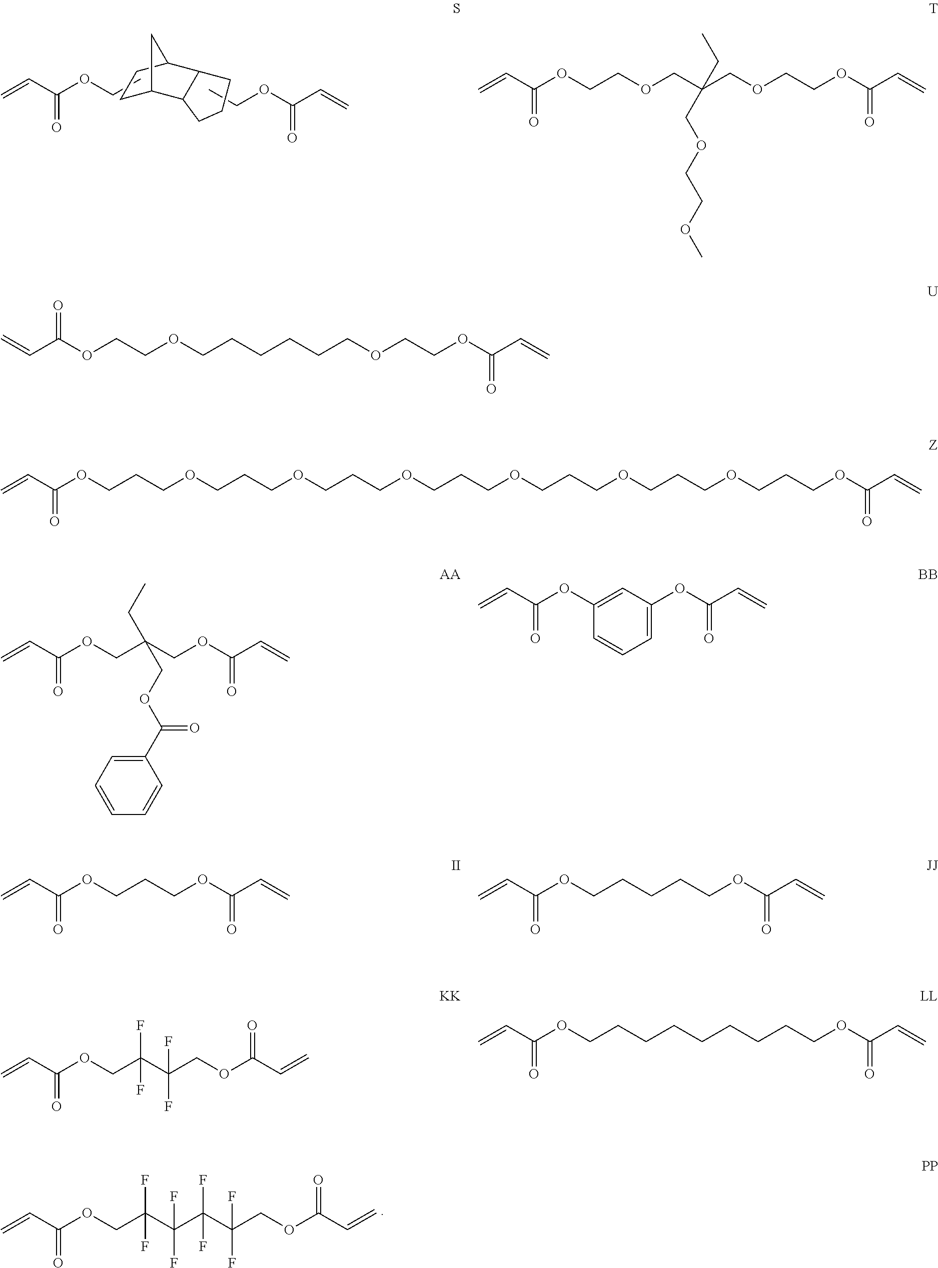

In some embodiments, the therapeutic agent is a therapeutic protein that includes Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In some embodiments, the poly(beta-amino ester) comprises a bis(acrylate ester) of the formula:

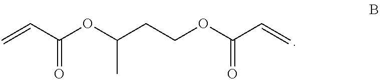

In some embodiments, the poly(beta-amino ester) comprises a bis(acrylate ester) of the formula:

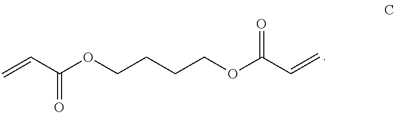

In some embodiments, the poly(beta-amino ester) comprises a bis(acrylate ester) of the formula:

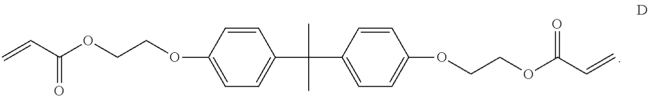

In some embodiments, the poly(beta-amino ester) comprises a bis(acrylate ester) of the formula:

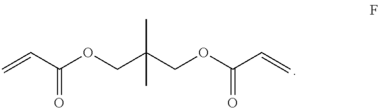

In some embodiments, the poly(beta-amino ester) comprises a bis(acrylate ester) of formula:

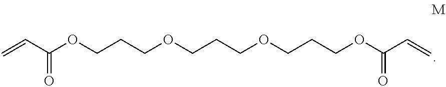

In some embodiments, the poly(beta-amino ester) comprises a bis(acrylate ester) of formula:

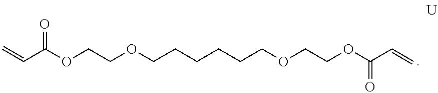

In some embodiments, the poly(beta-amino ester) comprises a bis(acrylate ester) of formula:

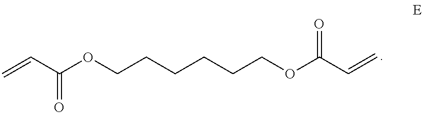

In some embodiments, the poly(beta-amino ester) comprises a bis(acrylate ester) of formula:

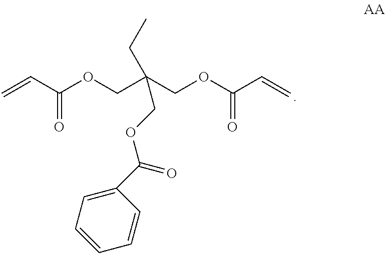

In some embodiments, the poly(beta-amino ester) comprises a bis(acrylate ester) of formula:

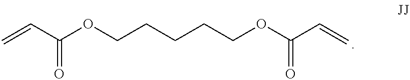

In some embodiments, the poly(beta-amino ester) comprises a bis(acrylate ester) of formula:

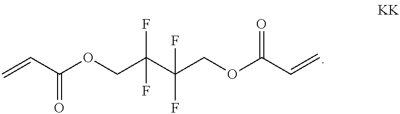

In some embodiments, the poly(beta-amino ester) comprises a bis(acrylate ester) of formula:

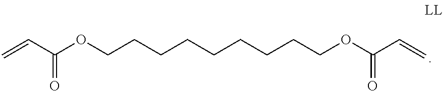

In some aspects, the disclosure features a therapeutic composition that contains a therapeutic agent (e.g., a therapeutic protein, e.g., a protein for use in replacement therapy, e.g., a protein described herein) and a poly(beta-amino ester) comprising an amine selected from the group consisting of the formulae 1-94:

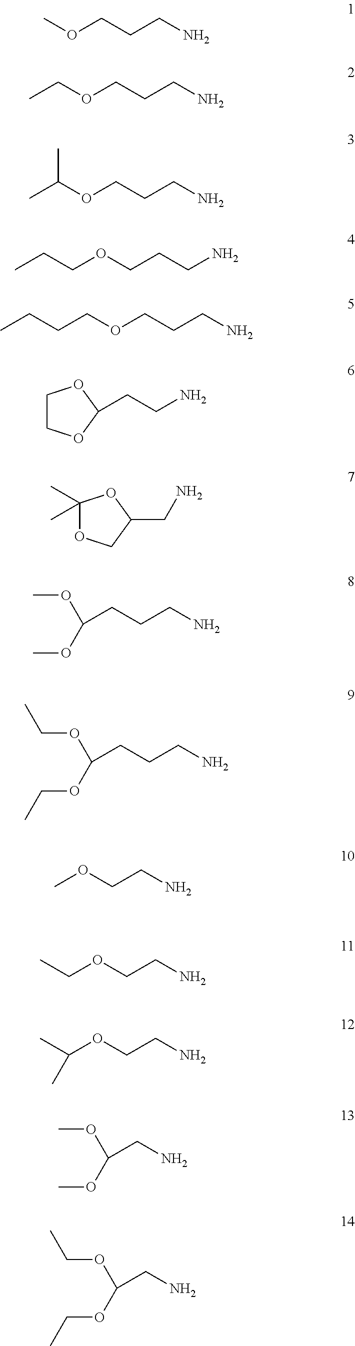

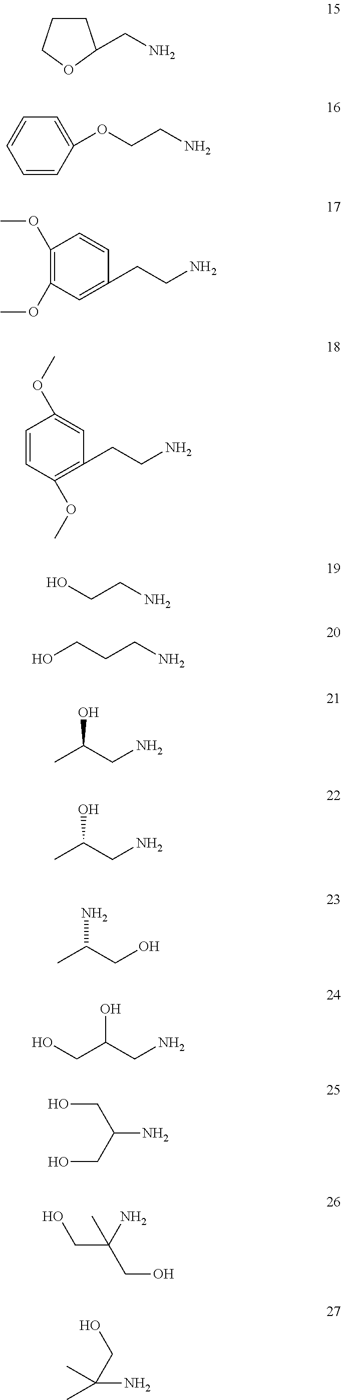

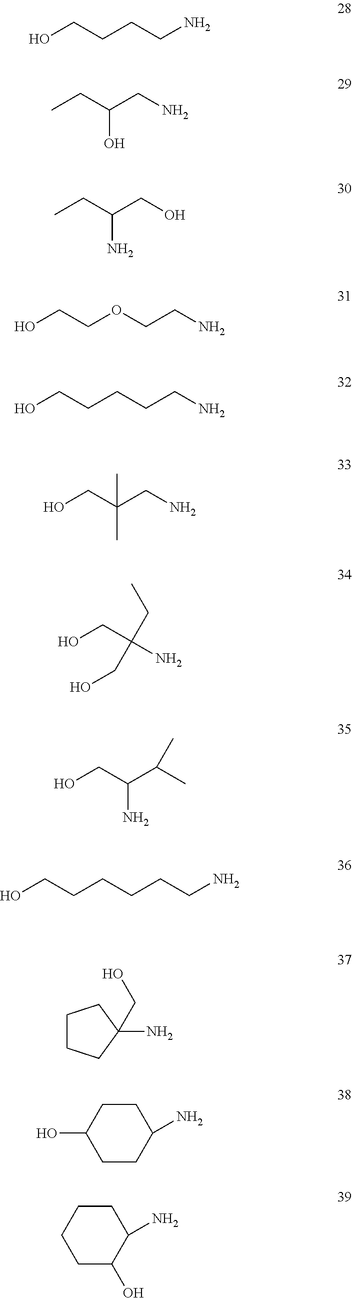

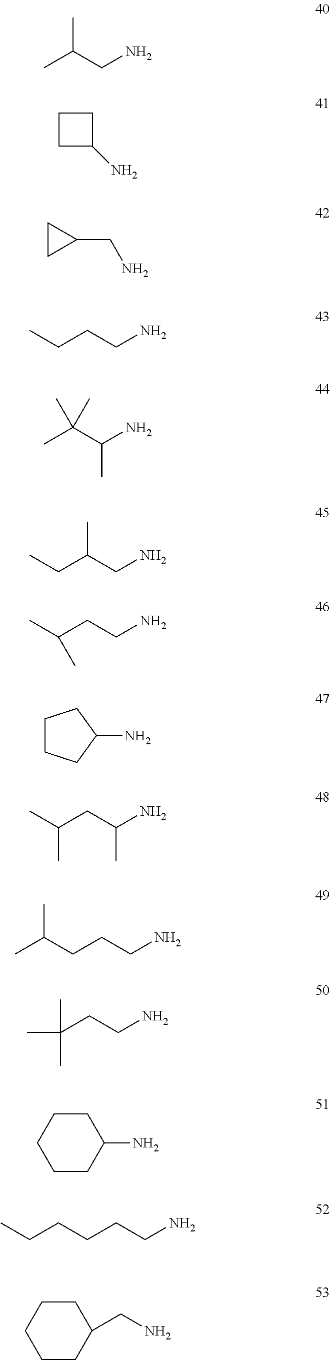

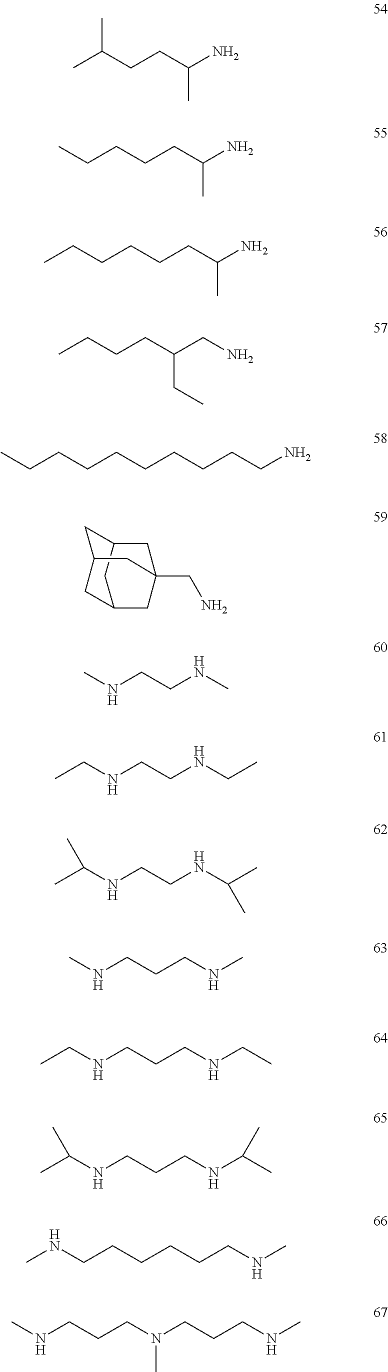

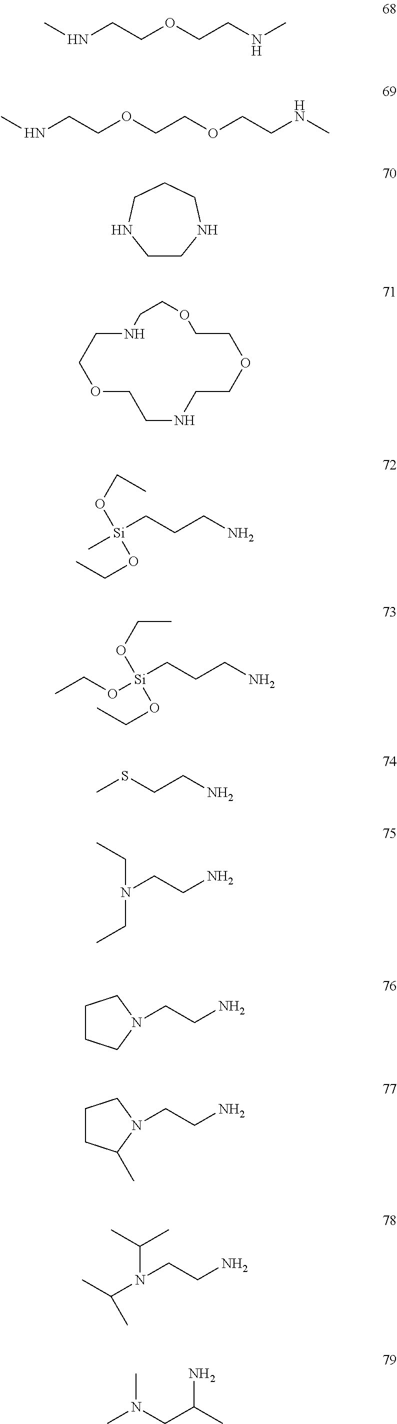

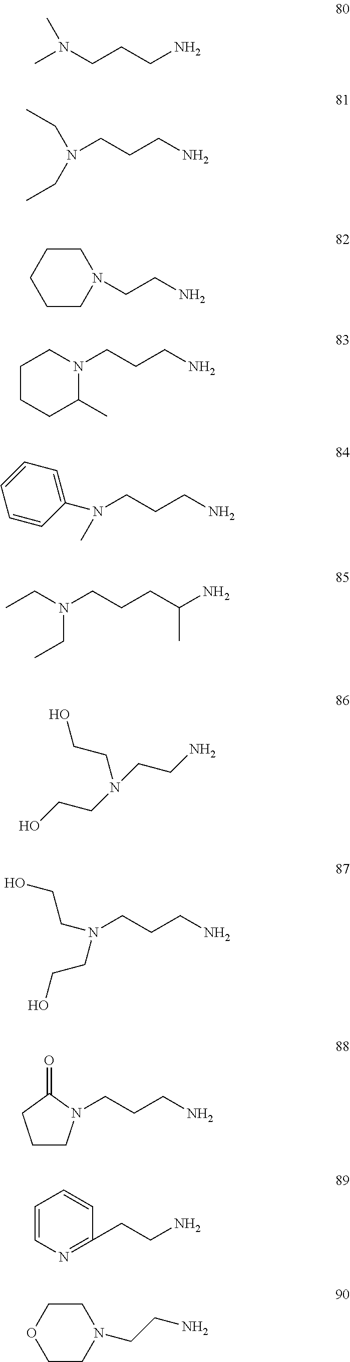

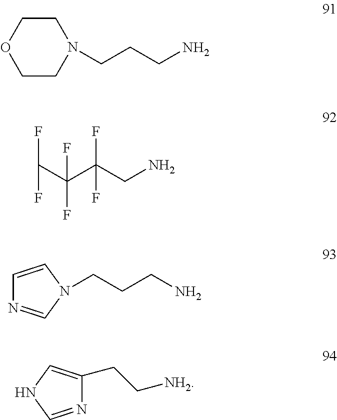

In some embodiments, the therapeutic agent is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In some embodiments, the poly(beta-amino ester) comprises an amine of formula:

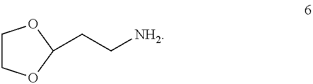

In some embodiments, the poly(beta-amino ester) comprises an amine of formula:

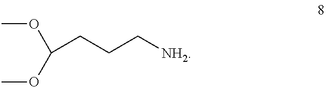

In some embodiments, the poly(beta-amino ester) comprises an amine of the formula:

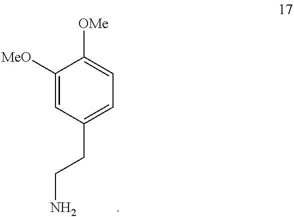

In some embodiments, the poly(beta-amino ester) comprises an amine of formula:

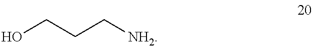

In some embodiments, the poly(beta-amino ester) comprises an amine of formula:

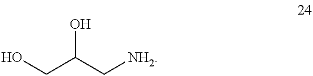

In some embodiments, the poly(beta-amino ester) comprises an amine of formula:

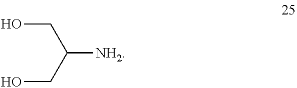

In some embodiments, the poly(beta-amino ester) comprises an amine of formula:

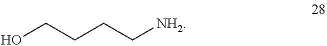

In some embodiments, the poly(beta-amino ester) comprises an amine of formula:

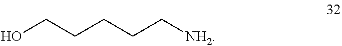

In some embodiments, the poly(beta-amino ester) comprises an amine of formula:

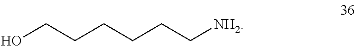

In some embodiments, the poly(beta-amino ester) comprises an amine of formula:

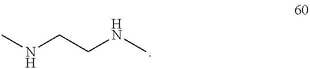

In some embodiments, the poly(beta-amino ester) comprises an amine of formula:

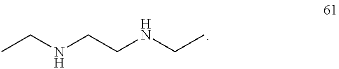

In some embodiments, the poly(beta-amino ester) comprises an amine of formula:

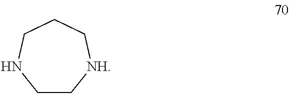

In some embodiments, the poly(beta-amino ester) comprises an amine of formula:

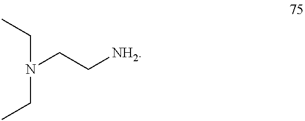

In some embodiments, the poly(beta-amino ester) comprises an amine of formula:

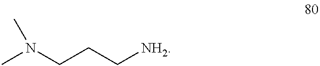

In some embodiments, the poly(beta-amino ester) comprises an amine of formula:

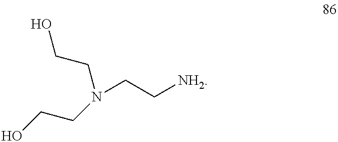

In some embodiments, the poly(beta-amino ester) comprises an amine of formula:

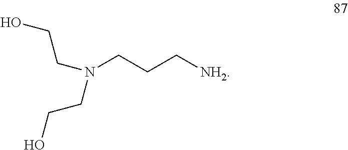

In some embodiments, the poly(beta-amino ester) comprises an amine of formula:

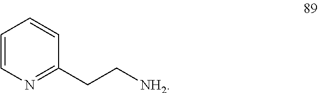

In some embodiments, the poly(beta-amino ester) comprises an amine of formula:

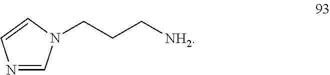

In some embodiments, the poly(beta-amino ester) comprises an amine of formula:

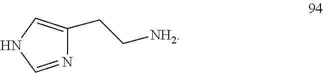

In other aspects, the disclosure features a pharmaceutical composition comprising a therapeutic composition described herein.

In other aspects, the disclosure features a pharmaceutical composition comprising nanoparticles containing a therapeutic composition described herein.

In other aspects, the disclosure features a pharmaceutical composition comprising microparticles containing a therapeutic composition described herein encapsulated in a matrix.

In some embodiments, the microparticles have a mean diameter of 1-10 micrometers.

In some embodiments, the microparticles have a mean diameter of less than 5 micrometers.

In some embodiments, the microparticles have a mean diameter of less than 1 micrometer.

In other aspects, the disclosure features a method of making a therapeutic composition, the method includes:
providing a poly(beta-amino ester) described herein;
providing a therapeutic agent described herein (e.g., a therapeutic protein, e.g., a protein for use in replacement therapy, e.g., a protein described herein); and
combining the poly(beta-amino ester) and the therapeutic agent, thereby making a therapeutic composition.

In some embodiments, the therapeutic agent is a therapeutic protein that includes Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In other aspects, the disclosure features a method of treating an individual, the method includes administering a therapeutic composition described herein to an individual in need of such treatment (e.g., an individual in need of replacement therapy).

In some embodiments, the therapeutic composition includes a therapeutic agent that is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In other aspects, the disclosure features a method of preparing microparticles, the method includes:

contacting a therapeutic agent described herein (e.g., a therapeutic protein, e.g., a protein for use in replacement therapy, e.g., a protein described herein) with a poly(beta-amino ester) herein in the presence of a solvent to form a mixture; and spray drying the mixture, thereby preparing microparticles.

In some embodiments, the invention features the use of a therapeutic composition described herein for use in therapy.

In some embodiments, the invention features the use of a therapeutic composition described herein for the preparation of a medicament for replacement therapy.

In some embodiments, the polymers are useful in preparing drug delivery devices for the therapeutic agent (e.g., a therapeutic protein, e.g., a protein for use in replacement therapy, e.g., a protein described herein) and pharmaceutical compositions thereof. The invention also provides methods of preparing particles (e.g., microspheres) containing the therapeutic agent (e.g., a protein for use in replacement therapy, e.g., a protein described herein) and other pharmaceutical compositions containing the polymers and a therapeutic agent (e.g., a therapeutic protein, e.g., a protein for use in replacement therapy, e.g., a protein described herein).

In some embodiments, the polymers are poly(beta-amino esters) or salts or derivatives thereof. Preferred polymers are biodegradable and biocompatible.

In some embodiments, the polymers have one or more tertiary amines in the backbone of the polymer. Preferred polymers have one or two tertiary amines per repeating backbone unit. The polymers may also be co-polymers in which one of the components is a poly(beta-amino ester). The polymers may be prepared, e.g., by condensing bis(secondary amines) or primary amines with bis(acrylate esters). A polymer is represented by either of the formulae below:

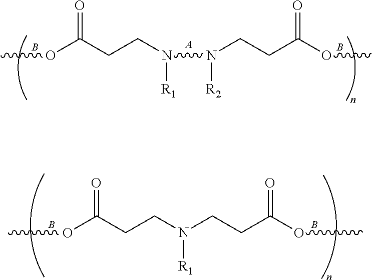

where A and B are linkers which may be any substituted or unsubstituted, branched or unbranched chain of carbon atoms or heteroatoms. The molecular weights of the polymers may range from about 1000 g/mol to about 20,000 g/mol, preferably from about 5000 g/mol to about 15,000 g/mol. In certain embodiments, B is an alkyl chain of one to twelve carbons atoms. In other embodiments, B is a heteroaliphatic chain containing a total of one to twelve carbon atoms and heteroatoms. The groups R1 and R2 may be any of a wide variety of substituents. In certain embodiments, R, and R2 may contain primary amines, secondary amines, tertiary amines, hydroxyl groups, and alkoxy groups. In certain embodiments, the polymers are amine-terminated; and in other embodiments, the polymers are acrylate-terminated. In a particularly preferred embodiment, the groups R1 and/or R2 form cyclic structures with the linker A (see the Detailed Description section below). Polymers containing such cyclic moieties have the characteristic of being more soluble at lower pH. Specifically preferred polymers are those that are insoluble in aqueous solutions at physiologic pH (pH 7.2-7.4) and are soluble in aqueous solutions below physiologic pH (pH<7.2). Other preferred polymers are those that are soluble in aqueous solution at physiologic pH (pH 7.2-7.4) and below.

The polymers can be prepared using commercially available or readily available monomers, bis(secondary amines), primary amines, and bis(acrylate esters), that are subjected to conditions which lead to the conjugate addition of the amine to the bis(acrylate ester). In a preferred embodiment, each of the monomers is dissolved in an organic solvent (e.g., DMSO, DMF, THF, diethyl ether (e.g., NMP diethyl ether), methylene chloride, hexanes, etc.), and the resulting solutions are combined and heated for a time sufficient to lead to polymerization of the monomers. In other embodiments, the polymerization is carried out in the absence of solvent. The resulting polymer may then be purified and optionally characterized using techniques known in the art.

In yet another aspect, the polymers are used to form nanometer-scale complexes with a therapeutic agent (e.g., a therapeutic protein, e.g., a protein for use in replacement therapy, e.g., a protein described herein). The therapeutic agent/polymer complexes may be formed by adding a solution of a therapeutic agent (e.g., a therapeutic protein, e.g., a protein for use in replacement therapy, e.g., a protein described herein) to a vortexing solution of the polymer at a desired therapeutic agent/polymer concentration. The weight to weight ratio of therapeutic agent to polymer may range from about 1:0.1 to about 1:200, preferably from 1:10 to 1:150, more preferably from 1:50 to 1:150. The amine monomer to therapeutic agent ratio may be approximately 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 110:1, 120:1, 130:1, 140:1, 150:1, 160:1, 170:1, 180:1, 190:1, and 200:1. Cationic polymers can be used that condense the therapeutic agent into soluble particles, e.g., 50-500 nm in size. These therapeutic agent/polymer complexes (e.g., therapeutic compositions) may be used in the delivery of a therapeutic agent (e.g., a therapeutic protein, e.g., a protein for use in replacement therapy, e.g., a protein described herein) to cells. In a particularly preferred embodiment, these complexes are combined with pharmaceutical excipients to form pharmaceutical compositions suitable for delivery to animals including humans.

In another aspect, the polymers are used to encapsulate therapeutic agents (e.g., a therapeutic protein, e.g., a protein for use in replacement therapy, e.g., a protein described herein) to form microparticles. The microparticles can range from about 1 micrometer to about 500 micrometers. In a particularly preferred embodiment, these microparticles allow for the delivery of the therapeutic agent to an individual. The microparticles may be prepared using any of the techniques known in the art to make microparticles, such as, for example, double emulsion, phase inversion, and spray drying. In a particularly preferred embodiment, the microparticles can be used for pH-triggered delivery of the encapsulated contents due to the pH responsive nature of the polymers (i.e., being more soluble at lower pH).

### DEFINITIONS

The following are chemical terms used in the specification and claims:

The term acyl as used herein refers to a group having the general formula —C(═O)R, where R is alkyl, alkenyl, alkynyl, aryl, carbocylic, heterocyclic, or aromatic heterocyclic. An example of an acyl group is acetyl.

The term alkyl as used herein refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-10 carbon atoms. In another embodiment, the alkyl group employed contains 1-8 carbon atoms. In still other embodiments, the alkyl group contains 1-6 carbon atoms. In yet another embodiments, the alkyl group contains 1-4 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substitutents.

The term alkoxy as used herein refers to a saturated (i.e., alkyl-O—) or unsaturated (i.e., alkenyl-O— and alkynyl-O—) group attached to the parent molecular moiety through an oxygen atom. In certain embodiments, the alkyl group contains 1-20 aliphatic carbon atoms. In certain other embodiments, the alkyl, akenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples include, but are not limited to, methoxy, ethoxy, propoxy, isopropoxy, n-butoxy, tert-butoxy, i-butoxy, sec-butoxy, neopentoxy, n-hexoxy, and the like.

The term alkenyl denotes a monovalent group derived from a hydrocarbon moiety having at least one carbon-carbon double bond by the removal of a single hydrogen atom. In certain embodiments, the alkenyl group employed in the invention contains 1-20 carbon atoms. In some embodiments, the alkenyl group employed in the invention contains 1-10 carbon atoms. In another embodiment, the alkenyl group employed contains 1-8 carbon atoms. In still other embodiments, the alkenyl group contains 1-6 carbon atoms. In yet another embodiments, the alkenyl group contains 1-4 carbons. Alkenyl groups include, for example, ethenyl, propenyl, butenyl, 1-methyl-2-buten-1-yl, and the like.

The term alkynyl as used herein refers to a monovalent group derived from a hydrocarbon having at least one carbon-carbon triple bond by the removal of a single hydrogen atom. In certain embodiments, the alkynyl group employed in the invention contains 1-20 carbon atoms. In some embodiments, the alkynyl group employed in the invention contains 1-10 carbon atoms. In another embodiment, the alkynyl group employed contains 1-8 carbon atoms. In still other embodiments, the alkynyl group contains 1-6 carbon atoms. Representative alkynyl groups include, but are not limited to, ethynyl, 2-propynyl(propargyl), 1-propynyl, and the like.

The term alkylamino, dialkylamino, and trialkylamino as used herein refers to one, two, or three, respectively, alkyl groups, as previously defined, attached to the parent molecular moiety through a nitrogen atom. The term alkylamino refers to a group having the structure —NHR′ wherein R′ is an alkyl group, as previously defined; and the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R″ are each independently selected from the group consisting of alkyl groups. In certain embodiments, the alkyl group contain 1-20 aliphatic carbon atoms. In certain other embodiments, the alkyl group contains 1-10 aliphatic carbon atoms. In yet other embodiments, the alkyl group contains 1-8 aliphatic carbon atoms. In still other embodiments, the alkyl group contain 1-6 aliphatic carbon atoms. In yet other embodiments, the alkyl group contain 1-4 aliphatic carbon atoms. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of thioalkoxyl moieties include, but are not limited to, methylthio, ethylthio, propylthio, isopropylthio, n-butylthio, and the like.

The term aryl as used herein refers to an unsaturated cyclic moiety comprising at least one aromatic ring. In certain embodiments, aryl group refers to a mono- or bicyclic carbocyclic ring system having one or two aromatic rings including, but not limited to, phenyl, naphthyl, tetrahydronaphthyl, indanyl, indenyl, and the like. Aryl groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide. In addition, substituted aryl groups include tetrafluorophenyl and pentafluorophenyl.

The term carboxylic acid as used herein refers to a group of formula —CO₂H.

The terms halo and halogen as used herein refer to an atom selected from fluorine, chlorine, bromine, and iodine.

The term heterocyclic, as used herein, refers to an aromatic or non-aromatic, partially unsaturated or fully saturated, 3- to 10-membered ring system, which includes single rings of 3 to 8 atoms in size and bi- and tri-cyclic ring systems which may include aromatic five- or six-membered aryl or aromatic heterocyclic groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring.

The term aromatic heterocyclic, as used herein, refers to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. Aromatic heterocyclic groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide.

Specific heterocyclic and aromatic heterocyclic groups that may be included in the compounds include: 3-methyl-4-(3-methylphenyl)piperazine, 3-methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, 4-(phenylmethyl)piperazine, 4-(1-phenylethyl)piperazine, 4-(1,1-dimethylethoxycarbonyl)piperazine, 4-(2-(bis-(2-propenyl)amino)ethyl)piperazine, 4-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl)piperazine, 4-(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl) carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

The term carbamoyl, as used herein, refers to an amide group of the formula —CONH₂.

The term carbonyldioxyl, as used herein, refers to a carbonate group of the formula —O—CO—OR.

The term hydrocarbon, as used herein, refers to any chemical group comprising hydrogen and carbon. The hydrocarbon may be substituted or unsubstituted. The hydrocarbon may be unsaturated, saturated, branched, unbranched, cyclic, polycyclic, or heterocyclic. Illustrative hydrocarbons include, for example, methyl, ethyl, n-propyl, iso-propyl, cyclopropyl, allyl, vinyl, n-butyl, tert-butyl, ethynyl, cyclohexyl, methoxy, diethylamino, and the like. As would be known to one skilled in this art, all valencies must be satisfied in making any substitutions.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

The term thiohydroxyl or thiol, as used herein, refers to a group of the formula SH.

The term ureido, as used herein, refers to a urea group of the formula: —NH—CO—NH₂.

The following are more general terms used throughout the present specification:

“Animal”: The term animal, as used herein, refers to humans as well as non-human animals, including, for example, mammals, birds, reptiles, amphibians, and fish.

Preferably, the non-human animal is a mammal (e.g., a rodent, a mouse, a rat, a rabbit, a monkey, a dog, a cat, a primate, or a pig). An animal may be a domesticated animal. An animal may be a transgenic animal.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Biocompatible”: The term “biocompatible”, as used herein is intended to describe compounds that are not toxic to cells. Compounds are “biocompatible” if their addition to cells in vitro results in less than or equal to 20% cell death, and their administration in vivo does not induce sustained inflammation or other such adverse effects.

“Biodegradable”: As used herein, “biodegradable” compounds are those that, when introduced into cells, are broken down by the cellular machinery, intra-intermolecular interactions or by hydrolysis into components that the cells can either reuse or dispose of without significant toxic effect on the cells (i.e., fewer than about 20% of the cells are killed when the components are added to cells in vitro). The components preferably do not induce inflammation or other adverse effects in vivo. In certain preferred embodiments, the chemical reactions relied upon to break down the biodegradable compounds are uncatalyzed.

“Effective amount”: In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, etc. For example, the effective amount of microparticles containing an antigen to be delivered to immunize an individual is the amount that results in an immune response sufficient to prevent infection with an organism having the administered antigen.

“Isolated composition”: An “isolated composition” refers to a composition that is removed from at least 90% of at least one component of a natural sample from which the isolated composition can be obtained. Compositions produced artificially or naturally can be “compositions of at least” a certain degree of purity if the species or population of species of interest is at least 5, 10, 25, 50, 75, 80, 90, 95, 96, 97, 98, or 99% pure on a weight-weight basis. For example, a therapeutic protein described herein can be an isolated therapeutic protein, or a therapeutic protein of at least a certain degree of purity. Similarly, a therapeutic composition (e.g., containing a therapeutic protein and poly(beta-amino ester)) described herein can be an isolated therapeutic composition, or a therapeutic composition of at least a certain degree of purity.

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

All cited publications, patent applications, patents, and other references mentioned herein are incorporated by reference in their entirety. In case of conflict, the present specification, including definitions, will control. In addition, the materials and methods are illustrative only and not intended to be limiting.

The details of one or more embodiments are set forth in the description below. Other features, objects, and advantages of the invention will be apparent from the description and from the claims.

### DETAILED DESCRIPTION

The present invention provides therapeutic compositions that contain a polymer (e.g., a poly(beta-amino ester) polymer) and a therapeutic agent (e.g., a therapeutic protein, e.g., a protein for use in replacement therapy, e.g., a protein described herein). Also described are polymeric encapsulation and delivery systems based on the use of poly(beta-amino ester) polymers. The systems may be used to prepare pharmaceutical compositions, e.g., that contain a therapeutic agent (e.g., a therapeutic protein, e.g., a protein for use in replacement therapy, e.g., a protein described herein), that can be administered to individuals in need of such administration (e.g., an individual with a protein deficiency or with low levels of the protein).

The tertiary amine-containing backbones of the polymers may be used to complex a therapeutic agent (e.g., a therapeutic protein, e.g., a protein for use in replacement therapy, e.g., a protein described herein) and thereby enhance the delivery of agent and prevent its degradation. The polymers may also be used in the formation of nanoparticles or microparticles containing encapsulated agents.

Due to the polymers' properties of being biocompatible and biodegradable, these formed therapeutic compositions, pharmaceutical compositions, particles (e.g., microparticles) are also biodegradable and biocompatible and may be used to provide controlled, sustained release of the encapsulated therapeutic agent. The particles may also be responsive to pH changes given the fact that these polymers are typically not substantially soluble in aqueous solution at physiologic pH but are more soluble at lower pH. The particles may also be responsive to thermal changes, e.g., viscosity changes at 37° C. (PLURONIC® copolymers (e.g., from BASF)). The particles may also be responsive to cationic additives, e.g., viscosity changes of alginate in the presence of divalent calcium. The particles may also be caused to form depot formulations upon administration to the body, e.g., technology from Atrix Laboratories or SABER™ technology from Durect Corporation.

Therapeutic Proteins

The poly(beta-amino esters) and polymers thereof described herein can be used to prepare a therapeutic composition that contains a therapeutic protein, e.g., a protein for replacement therapy. The protein can be, e.g., an enzyme. Examples of proteins that can be used for replacement therapy include: Bruton's tyrosine kinase (BTK), ornithine transcarbamylase (OTC), survival motor neuron protein (SMN1 or SMN2), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, N-acetylgalactosamine-4-sulfatase, protein C, protein S, CLN2 (ceroid lipofuscinosis, neuronal 2) protein, alpha-1 antitrypsin, Factor VIII, Factor IX, albumin, pancrelipase (e.g., CREON, Cotazym, Pancrease, Pancrease MT, ULTRASE®).

Preferred proteins for the compositions described herein include Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, and N-acetylgalactosamine-4-sulfatase.

Bruton's Tyrosine Kinase (BTK)

Bruton's tyrosine kinase is a type of kinase enzyme implicated in the primary immunodeficiency disease X-linked agammaglobulinemia (XLA). It plays a crucial role in B cell maturation as well as mast cell activation through the high-affinity IgE receptor. Patients with XLA have normal pre-B cell populations in their bone marrow but these cells fail to mature and enter the circulation. The BTK gene is located on the X chromosome. At least 24 mutations of the BTK gene have been identified.

BTK contains a PH domain which binds Phosphatidylinositol (3,4,5)-trisphosphate (PIP3). PIP3 binding induces BTK to phosphorylate phospholipase C, which in turn hydrolyzes PIP2 into two second messengers, inositol triphosphate (IP3) and diacylglycerol (DAG), which then go on to modulate the activity of downstream proteins during B-cell signalling.

A therapeutic composition that contains BTK and a polymer described herein, and the methods described herein, can be used in the treatment of an individual with a need for BTK replacement therapy.

Ornithine Transcarbamylase (OTC)

Ornithine transcarbamylase deficiency (OTC) is a rare metabolic disorder. It is a genetic disorder resulting in a mutated and ineffective form of the enzyme ornithine transcarbamylase.

OTC affects the body's ability to get rid of ammonia. As a result, ammonia accumulates in the blood causing hyperammonemia. This ammonia travels to the various organs of the body including the brain, causing coma, brain damage, liver damage, and death.

Ornithine transcarbamylase deficiency often becomes evident in the first few days of life. An infant with ornithine transcarbamylase deficiency may be lethargic or unwilling to eat, and have poorly-controlled breathing rate or body temperature. Some babies with this disorder may experience seizures or unusual body movements, or go into a coma. Complications from ornithine transcarbamylase deficiency may include developmental delay and mental retardation. Progressive liver damage, skin lesions, and brittle hair may also be seen. Other symptoms include irrational behavior (caused by encephalitis), mood swings, and poor performance in school.

Mutations in the OTC gene cause ornithine transcarbamylase deficiency. Ornithine transcarbamylase deficiency is an X-linked disorder caused by a number of different mutations. Since the gene is on the X chromosome, females are primarily carriers while males with nonconservative mutations rarely survive past 72 hours of birth. Half of those survivors die in the first month, and half of the remaining by age 5.

A therapeutic composition that contains OTC and a polymer described herein, and the methods described herein, can be used in the treatment of an individual with a need for OTC replacement therapy.

Survival Motor Neuron Protein (SMN)

Loss of the SMN1 gene causes Spinal Muscular Atrophy (SMA), which manifests as weakness, due to loss of the motor neurons of the spinal cord and brainstem.

The region of chromosome 5 that contains the SMN (Survival Motor Neuron) gene has a large duplication. A large sequence that contains several genes occurs twice in adjacent segments. There are thus two copies of the gene, SMN1 and SMN2. The SMN2 gene has an additional mutation that makes it less efficient at making protein, though it does so in a low level. SMA is caused by loss of the SMN1 gene from both chromosomes. The severity of SMA, ranging from SMA 1 to SMA 3, is partly related to how well the remaining SMN 2 genes can make up for the loss of SMN 1. Often there are additional copies of SMN2, and an increasing number of SMN2 copies causes less severe disease.

Infantile SMA is the most severe form. Some of the symptoms include: muscle weakness, poor muscle tone, weak cry, limpness or a tendency to flop, difficulty sucking or swallowing, accumulation of secretions in the lungs or throat, the legs tend to be weaker than the arms, feeding difficulties, increased susceptibility to respiratory tract infections, developmental milestones, such as lifting the head or sitting up, can't be reached. Although SMA often results in death during childhood, some people with SMA survive into adulthood and even old age. Actual lifespan depends greatly on the severity of SMA in each individual.

In general, the earlier the symptoms appear, the shorter the life span. The onset is sudden and dramatic. Once symptoms appear the motor neuron cells quickly deteriorate shortly after. The disease can be fatal and there is no cure for SMA yet known. The major management issue in Type 1 SMA is the prevention and early treatment of respiratory infections; pneumonia is the cause of death in the majority of the cases. Infants with Type 1 SMA have a life expectancy of less than two years, however, some grow to be adults. Intellectual and later, sexual functions, are unaffected by SMA.

A therapeutic composition that contains SMN and a polymer described herein, and the methods described herein, can be used in the treatment of an individual with a need for SMN replacement therapy.

Galactocerebrosidase (GALC)

Krabbe's Leukodystrophy is a rare inherited lipid storage disorder caused by a deficiency of the enzyme galactocerebrosidase (GALC), which is necessary for the metabolism of the sphingolipids galactosylceremide and psychosine. Failure to break down these sphingolipids results in degeneration of the myelin sheath surrounding nerves in the brain (demyelination). Characteristic globoid cells appear in affected areas of the brain. This metabolic disorder is characterized by progressive neurological dysfunction such as mental retardation, paralysis, blindness, deafness and paralysis of certain facial muscles (pseudobulbar palsy). Krabbe's Leukodystrophy is inherited as an autosomal recessive trait.

A therapeutic composition that contains GALC and a polymer described herein, and the methods described herein, can be used in the treatment of an individual with a need for GALC replacement therapy.

N-Sulfoglucosamine Sulfohydrolase

MPS type III-A (Sanfilipo A syndrome) is a lysosomal storage disorder caused by deficiency or malfunction of N-sulfoglucosamine sulfohydrolase (also referred to as heparan sulfate sulfatase or sulfamidase), which is required for the degradation of heparan sulphate. Patients develop severe central nervous system degeneration resulting in progressive dementia often combined with delayed speech, sleep disturbance, hirsutism, diarrhoea, hyperactivity and aggressive behavior. Clinical features can also include severe mental defect with relatively mild somatic features (moderately severe claw hand and visceromegaly, little or no corneal clouding or skeletal, e.g., vertebral, change). The presenting problem may be marked overactivity, destructive tendencies, and other behavioral aberrations in a child of 4 to 6 years of age. Clinical onset in severely affected patients usually occurs following 2-3 years of apparently normal development. Mild skeletal pathology, joint stiffness and hepatosplenomegaly are often present in older patients. Patients may present and develop within a wide spectrum of clinical severity. The radiologic findings in the skeleton are relatively mild and include persistent biconvexity of the vertebral bodies and very thick calvaria.

A therapeutic composition that contains N-sulfoglucosamine sulfohydrolase and a polymer described herein, and the methods described herein, can be used in the treatment of an individual with a need for N-sulfoglucosamine sulfohydrolase replacement therapy.

### N-acetylglucosaminidase

The Sanfilippo syndrome type B is a lysosomal storage disorder caused by deficiency of alpha-N-acetylglucosaminidase; it is characterized by profound mental deterioration in childhood and death in the second decade. The cDNA sequence was found to encode a protein of 743 amino acids, with a 20- to 23-aa signal peptide immediately preceding the amino terminus of the tissue enzyme and with six potential N-glycosylation sites. The 8.5-kb gene (NAGLU), interrupted by 5 introns, was localized to the 5′-flanking sequence of a known gene, EDH17B, on chromosome 17q21. Five mutations have been identified in cells of patients with Sanfilippo syndrome type B: 503del10, R297X, R626X, R643H, and R674H. The occurrence of a frameshift and a nonsense mutation in homozygous form confirms the identity of the NAGLU gene.

A therapeutic composition that contains N-acetylglucosaminidase and a polymer described herein, and the methods described herein, can be used in the treatment of an individual with a need for N-acetylglucosaminidase replacement therapy.

### Iduronate-2-sulfatase

Hunter syndrome, or mucopolysaccharoidosis Type II (MPS II), is a lysosomal storage disease caused by a deficiency of iduronate-2-sulfatase (I2S). The I2S gene is located on the X chromosome. Hunter syndrome is a serious genetic disorder that primarily affects males. It interferes with the body's ability to break down and recycle specific glycosaminoglycans or GAG. Hunter syndrome is one of several related lysosomal storage diseases.

In Hunter syndrome, GAG build up in cells throughout the body due to a deficiency or absence of the enzyme iduronate-2-sulfatase (I2S). This buildup interferes with the way certain cells and organs in the body function and leads to a number of serious symptoms. As the buildup of GAG continues throughout the cells of the body, signs of Hunter syndrome become more visible. Physical manifestations for some people with Hunter syndrome include distinct facial features, a large head, and an enlarged abdomen. People with Hunter syndrome may also experience hearing loss, thickening of the heart valves leading to a decline in cardiac function, obstructive airway disease, sleep apnea, and enlargement of the liver and spleen. Range of motion and mobility may also be affected. In some cases of Hunter syndrome, central nervous system involvement leads to developmental delays and nervous system problems. Not all people with Hunter syndrome are affected by the disease in exactly the same way, and the rate of symptom progression varies widely. However, Hunter syndrome is always severe, progressive, and life-limiting.

ELAPRASE™ is a synthetic version of I2S that was approved by the United States Food and Drug Administration for enzyme replacement treatment for Hunter syndrome.

A therapeutic composition that contains I2S and a polymer described herein, and the methods described herein, can be used in the treatment of an individual with a need for I2S replacement therapy.

Alpha-Glucosidase

Pompe disease (acid maltase deficiency (AMD), glycogen storage disease type II) is a rare, inherited and often fatal disorder that disables the heart and muscles. It is caused by mutations in a gene that makes alpha-glucosidase (GAA). Normally, the body uses GAA to break down glycogen. But in Pompe disease, mutations in the GAA gene reduce or completely eliminate this essential enzyme. Excessive amounts of glycogen accumulate everywhere in the body, but the cells of the heart and skeletal muscles are the most seriously affected. Researchers have identified up to 70 different mutations in the GAA gene that cause the symptoms of Pompe disease, which can vary widely in terms of age of onset and severity. The severity of the disease and the age of onset are related to the degree of enzyme deficiency.

Early onset (or infantile) Pompe disease is the result of complete or near complete deficiency of GAA. Symptoms begin in the first months of life, with feeding problems, poor weight gain, muscle weakness, floppiness, and head lag. Respiratory difficulties are often complicated by lung infections. The heart is grossly enlarged. More than half of all infants with Pompe disease also have enlarged tongues. Most babies with Pompe disease die from cardiac or respiratory complications before their first birthday.

Late onset (or juvenile/adult) Pompe disease is the result of a partial deficiency of GAA. The onset can be as early as the first decade of childhood or as late as the sixth decade of adulthood. The primary symptom is muscle weakness progressing to respiratory weakness and death from respiratory failure after a course lasting several years. The heart may be involved but it will not be grossly enlarged. A diagnosis of Pompe disease can be confirmed by screening for the common genetic mutations or measuring the level of GAA enzyme activity in a blood sample—a test that has 100 percent accuracy. Once Pompe disease is diagnosed, testing of all family members and consultation with a professional geneticist is recommended. Carriers are most reliably identified via genetic mutation analysis.

A therapeutic composition that contains GAA and a polymer described herein, and the methods described herein, can be used in the treatment of an individual with a need for GAA replacement therapy.

Sulfatase-Modifying Factor 1 (SUMF1)

Sulfatase modifying factor 1 (SUMF1) is the gene mutated in multiple sulfatase deficiency (MSD) and encodes the formylglycine-generating enzyme, an essential activator of all the sulfatases. The disorder combines features of metachromatic leukodystrophy and of a mucopolysaccharidosis. Increased amounts of acid mucopolysaccharides are found in several tissues. In contrast to the classic form of metachromatic leukodystrophy, arylsulfatases A, B, and C are absent in the Austin type of juvenile sulfatidosis. The ‘gargoylism’ features are mild. Neurologic deterioration is rapid. Both mucopolysaccharide and sulfatide are found in the urine in excess. Cerebrospinal fluid protein is increased. Peripheral nerves show metachromatic degeneration of myelin on biopsy. The disease is associated with ichthyosis, dysostosis multiplex, psychomotor retardation, and coarse facies.

A therapeutic composition that contains SUMF1 and a polymer described herein, and the methods described herein, can be used in the treatment of an individual with a need for SUMF1 replacement therapy.

Glucocerebrosidase (GCB)

Gaucher patients exhibit a glucocerebrosidase deficiency. Glucocerebrosidase (also known as glucoceramidase) is involved in the breakdown and recycling of glucocerebroside. Gaucher disease is an inherited metabolic disorder in which harmful quantities of a fatty substance called glucocerebroside accumulate in the spleen, liver, lungs, bone marrow, and sometimes in the brain. There are three types of Gaucher disease. The first category, called type 1, is by far the most common. Patients in this group usually bruise easily and experience fatigue due to anemia and low blood platelets. They also have an enlarged liver and spleen, skeletal disorders, and, in some instances, lung and kidney impairment. There are no signs of brain involvement. Symptoms can appear at any age. In type 2 Gaucher disease, liver and spleen enlargement are apparent by 3 months of age. Patients have extensive and progressive brain damage and usually die by 2 years of age. In the third category, called type 3, liver and spleen enlargement is variable, and signs of brain involvement such as seizures gradually become apparent. The buildup of this fatty material within cells prevents the cells and organs from functioning properly.

Examples of GCB protein that can be used in the therapeutic compositions described herein include CEREZYME® (imiglucerase for injection; Genzyme Corporation), and the proteins described in, e.g., WO02/15927, WO2005/089047, WO03/056897, WO01/77307, WO01/07078, and WO90/07573; European Published App. No. EP1392826; U.S. Published Application Nos. 2005-0026249, 2005-0019861, 2002-0168750, 2005-0265988, 2004-0043457, 2003-0215435, and 2003-0133924; and U.S. patent application Ser. No. 10/968,870; U.S. Pat. Nos. 7,138,262, 6,451,600, 6,074,864, 5,879,680, 5,549,892, 5,236,838, and 3,910,822.

A therapeutic composition that contains GCB and a polymer described herein, and the methods described herein, can be used in the treatment of an individual with a need for GCB replacement therapy.

Alpha Galactosidase

An alpha galactosidase mutation causes Fabry disease. A mutation in the gene that controls this enzyme causes insufficient breakdown of lipids, which build up to harmful levels in the eyes, kidneys, autonomic nervous system, and cardiovascular system. Since the gene that is altered is carried on a mother's X chromosome, her sons have a 50 percent chance of inheriting the disorder and her daughters have a 50 percent chance of being a carrier. Symptoms usually begin during childhood or adolescence and include burning sensations in the hands that gets worse with exercise and hot weather and small, raised reddish-purple blemishes on the skin. Some boys will also have eye manifestations, especially cloudiness of the cornea. Lipid storage may lead to impaired arterial circulation and increased risk of heart attack or stroke. The heart may also become enlarged and the kidneys may become progressively involved. Other symptoms include decreased sweating, fever, and gastrointestinal difficulties, particularly after eating. Some female carriers may also exhibit symptoms. Fabry disease is one of several lipid storage disorders.

Examples of alpha-galactosidase protein that can be used in the therapeutic compositions described herein include those described in, e.g., WO98/11206 and WO00/53730.

A therapeutic composition that contains alpha-galactosidase and a polymer described herein, and the methods described herein, can be used in the treatment of an individual with a need for alpha-galactosidase replacement therapy.

Alpha Iduronidase

Alpha iduronidase (IDUA) deficiency is responsible for Hurler syndrome (also known as mucopolysaccharidosis type I (MPS I), Hurler's disease and gargoylism). Alpha-L iduronidase functions to break down mucopolysaccharides. Without this enzyme, the buildup of heparan sulfate and dermatan sulfate occurs in the body (the heart, liver, brain etc.). Symptoms appear during childhood and early death can occur due to organ damage.

MPS I is divided into three subtypes based on severity of symptoms. All three types result from an absence of, or insufficient levels of, the enzyme alpha-L-iduronidase. MPS I H or Hurler syndrome is the most severe of the MPS I subtypes. The other two types are MPS I S or Scheie syndrome and MPS I H-S or Hurler-Scheie syndrome. Hurler syndrome is marked by progressive deterioration, hepatosplenomegaly, dwarfism and gargoyle-like faces. There is a progressive mental retardation, with death occurring by the age of 10 years.

Developmental delay is evident by the end of the first year, and patients usually stop developing between ages 2 and 4. This is followed by progressive mental decline and loss of physical skills. Language may be limited due to hearing loss and an enlarged tongue. In time, the clear layers of the cornea become clouded and retinas may begin to degenerate. Carpal tunnel syndrome (or similar compression of nerves elsewhere in the body) and restricted joint movement are common.

Hurler's Syndrome is often classified as a lysosomal storage disease and is mechanistically related to Hunter's Syndrome (X-linked recessive). Children born to an MPS I parent carry a defective IDUA gene, which has been mapped to the 4p16.3 site on chromosome 4. As of 2001, 52 different mutations in the IDUA gene have been shown to cause Hurler syndrome. Because Hurler syndrome is an autosomal recessive disorder.

Therapeutic forms if IDUA include laronidase, e.g., ALDURAZYME®.

A therapeutic composition that contains IDUA and a polymer described herein, and the methods described herein, can be used in the treatment of an individual with a need for IDUA replacement therapy.

Beta Glucuronidase

Sly syndrome belongs to a group of disorders known as the mucopolysaccharidoses, which are lysosomal storage diseases. It is characterized by a deficiency of the enzyme β-glucuronidase, a lysosomal enzyme. In Sly syndrome, the deficiency in β-glucuronidase leads to the accumulation of certain complex carbohydrates (mucopolysaccharides) in many tissues and organs of the body. The defective gene lies on chromosome 7.

Sly syndrome is also known as Mucopolysaccahridosis Type VII (MPS), β-glucurondinase deficiency, β-glucurondinase deficiency mucopolysaccahridosis, GUSB deficiency, mucopolysaccahride storage disease VII, MCA, and MR.

The symptoms of Sly syndrome are similar to those of Hurler syndrome (MPS I). The symptoms include: in the head, neck, and face: coarse (Hurler-like) facies and macrocephaly, frontal prominence, premature closure of sagittal lambdoid sutures, and J-shaped sella turcica, in the eyes: corneal opacity and iris colobmata, in the nose: anteverted nostrils and a depressed nostril bridge, in the mouth and oral areas: prominent alveolar processes and cleft palate, in the thorax: usually pectus carinatum or exacavatum and oar-shaped ribs; also a protruding abdomen and inguinal or umbilical hernia, in the extremities: talipes, an underdeveloped ilium, aseptic necrosis of femoral head, and shortness of tubular bones occurs, in the spine: kyphosis or scoliosis and hook-like deformities in thoracic and lumbar vertebrate, in the bones: dysotosis multiplex. In addition, recurrent pulmonary infections occur. Hepatomegaly occurs in the gastrointestinal system. Splenomegaly occurs in the hematopoietic system. Inborn mucopolysaccharide metabolic disorders due to β-glucuronidase deficiency with granular inclusions in granulocytes occurs in the biochemical and metabolic systems. Growth and motor skills are affected, and mental retardation also occurs.

A therapeutic composition that contains beta glucuronidase and a polymer described herein, and the methods described herein, can be used in the treatment of an individual with a need for beta glucuronidase replacement therapy.

### N-acetylgalactosamine-4-sulfatase (arylsulfatase B)

Mucopolysacchariodosis type VI (MPS VI, Maroteaux-Lamy syndrome) is a lysosomal storage disorder caused by the deficient activity of N-acetylgalactosamine-4-sulfatase (arylsulfatase B) and the subsequent accumulation of the glycosaminoglycan (GAG) dermatan sulfate. Children with MPS VI usually have normal intellectual development but share many of the physical symptoms found in Hurler syndrome. The condition has a variable spectrum of severe symptoms. Neurological complications include clouded corneas, deafness, thickening of the dura (the membrane that surrounds and protects the brain and spinal cord), and pain caused by compressed or traumatized nerves and nerve roots. MPS VI is characterized by short stature, dysotosis multiplex, coarse facial features, cardiac valve anomalies, thickening of the tracheal wall.

Growth in children with the disorder is normal at first but stops suddenly around age 8. By age 10 children have developed a shortened trunk, crouched stance, and restricted joint movement. In more severe cases, children also develop a protruding abdomen and forward-curving spine. Skeletal changes (particularly in the pelvic region) are progressive and limit movement. Many children also have umbilical or inguinal hernias. Nearly all children have some form of heart disease, usually involving valve dysfunction.

A therapeutic composition that contains N-acetylgalactosamine-4-sulfatase and a polymer described herein, and the methods described herein, can be used in the treatment of an individual with a need for N-acetylgalactosamine-4-sulfatase replacement therapy.

Polymers

The therapeutic compositions of the present disclosure include a poly(beta-amino ester) and a therapeutic agent. The polymers are poly(beta-amino esters)-containing tertiary amines in their backbones and salts thereof. Such polymers are also described in WO 2004/106411. The molecular weights of the polymers may range from 5,000 g/mol to over 100,000 g/mol, more preferably from 4,000 g/mol to 50,000 g/mol. In a particularly preferred embodiment, the polymers are relatively non-cytotoxic. In another particularly preferred embodiment, the polymers are biocompatible and biodegradable. In a particularly preferred embodiment, the polymers have pKa's in the range of 5.5 to 7.5, more preferably between 6.0 and 7.0. In another particularly preferred embodiment, the polymer may be designed to have a desired pKa between 3.0 and 9.0, more preferably between 5.0 and 8.0. The polymers are particularly attractive for drug delivery for several reasons: 1) they contain amino groups for interacting with DNA and other negatively charged agents, for buffering the pH, for causing endosomolysis, etc.; 2) they contain degradable polyester linkages; 3) they can be synthesized from commercially available starting materials; and 4) they are pH responsive and future generations could be engineered with a desired pKa. In screening for transfection efficiency, the best performing polymers were hydrophobic or the diacrylate monomers were hydrophobic, and many had mono- or di-hydroxyl side chains and/or linear, bis(secondary amines) as part of their structure.

The polymers can generally be defined by the formula (I)

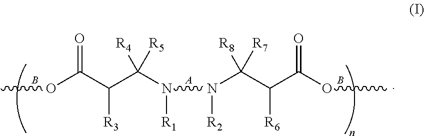

The linkers A and B are each a chain of atoms covalently linking the amino groups and ester groups, respectively. These linkers may contain carbon atoms or heteroatoms (e.g., nitrogen, oxygen, sulfur, etc.). Typically, these linkers are 1 to 30 atoms long, more preferably 1-15 atoms long. The linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted. The groups R1, R2, R3, R4, R5, R6, R7, and R8 may be any chemical groups including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, alkylthioether, thiol, and ureido groups. In the polymers, n is an integer ranging from 5 to 10,000, more preferably from 10 to 500.

In a particularly preferred embodiment, the bis(secondary amine) is a cyclic structure, and the polymer is generally represented by the formula II:

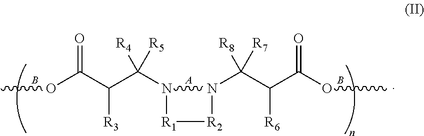

In this embodiment, R1 and R2 are directly linked together as shown in formula II.

Examples of polymers in this embodiment include, but are not limited to formulas III and IV:

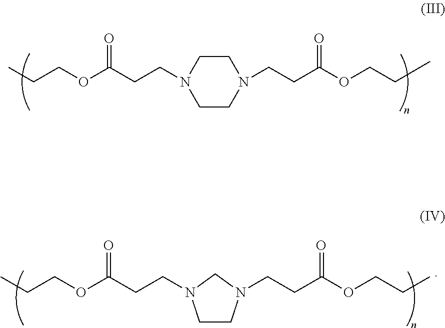

As described above in the preceding paragraph, any chemical group that satisfies the valency of each atom may be substituted for any hydrogen atom.

In another particularly preferred embodiment, the groups R1 and/or R2 are covalently bonded to linker A to form one or two cyclic structures. The polymers of the present embodiment are generally represented by the formula V in which both R1 and R2 are bonded to linker A to form two cyclic structures:

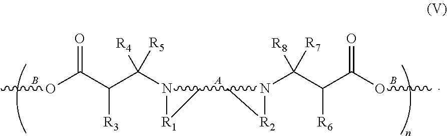

The cyclic structures may be 3-, 4-, 5-, 6-, 7-, or 8-membered rings or larger. The rings may contain heteroatoms and be unsaturated. Examples of polymers of formula V include formulas VI, VII, and VIII:

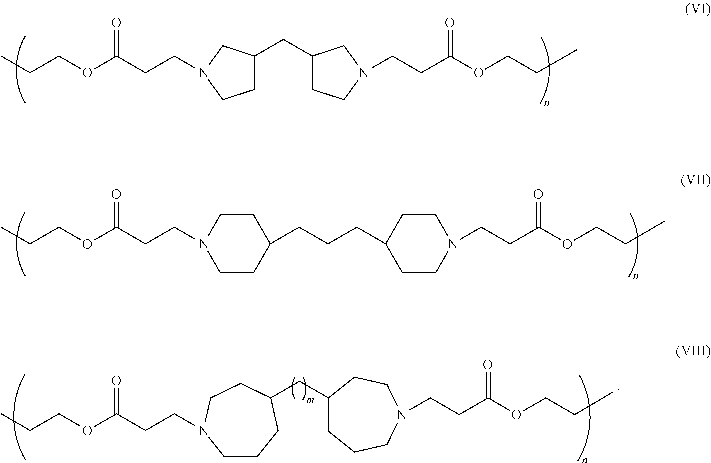

As described above, any chemical group that satisfies the valency of each atom in the molecule may be substituted for any hydrogen atom.

In another embodiment, the polymers can generally be defined by the formula (IX):

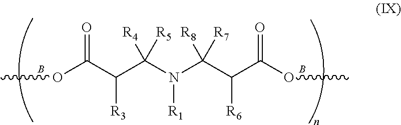

The linker B is a chain of atoms covalently linking the ester groups. The linker may contain carbon atoms or heteroatoms (e.g., nitrogen, oxygen, sulfur, etc.). Typically, the linker is 1 to 30 atoms long, preferably 1-15 atoms long, and more preferably 2-10 atoms long. In certain embodiments, the linker B is a substituted or unsubstituted, linear or branched alkyl chain, preferably with 3-10 carbon atoms, more preferably with 3, 4, 5, 6, or 7 carbon atoms. In other embodiments, the linker B is a substituted or unsubstituted, linear or branched heteroaliphatic chain, preferably with 3-10 atoms, more preferably with 3, 4, 5, 6, or 7 atoms. In certain embodiments, the linker B is comprises of repeating units of oxygen and carbon atoms. The linker may be substituted with various substituents including, but not limited to, hydrogen atoms, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, acyl, acetyl, and ureido groups.

As would be appreciated by one of skill in this art, each of these groups may in turn be substituted. Each of R1, R3, R4, R5, R6, R7, and R8 may be independently any chemical group including, but not limited to, hydrogen atom, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, alkylthioether, thiol, acyl, acetyl, and ureido groups. In certain embodiments, R1 includes hydroxyl groups. In other embodiments, R1 includes amino, alkylamino, or dialkylamino groups. In the polymer, n is an integer ranging from 5 to 10,000, more preferably from 10 to 500.

In certain embodiments, the polymers are generally defined as follows:

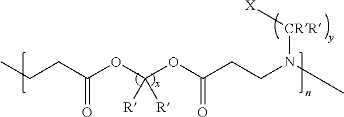

wherein

X is selected from the group consisting of C1-C6 lower alkyl, C1-C6 lower alkoxy, halogen, OR and NR2; more preferably, methyl, OH, or NH2;

R is selected from the group consisting of hydrogen, alkyl, alkenyl, alkynyl, cyclic, heterocyclic, aryl, and heteroaryl; each R′ is independently selected from the group consisting of hydrogen, C1-C6 lower alkyl, C1-C6 lower alkoxy, hydroxy, amino, alkylamino, dialkylamino, cyano, thiol, heteroaryl, aryl, phenyl, heterocyclic, carbocyclic, and halogen; preferably, R′ is hydrogen, methyl, ethyl, propyl, isopropyl, butyl, methoxy, ethoxy, propoxy, isopropoxy, hydroxyl, amino, fluoro, chloro, or bromo; more preferably, R′ is fluoro, hydrogen, or methyl; n is an integer between 3 and 10,000; x is an integer between 1 and 10; preferably, x is an integer between 2 and 6; y is an integer between 1 and 10; preferably, x is an integer between 2 and 6; and derivatives and salts thereof.

In certain embodiments, the polymers are generally defined as follows:

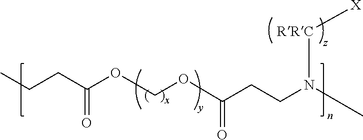

wherein

X is selected from the group consisting of C1-C6 lower alkyl, Ci-C6 lower alkoxy, halogen, OR and NR2; more preferably, methyl, OH, or NH2; R is selected from the group consisting of hydrogen, alkyl, alkenyl, alkynyl, cyclic, heterocyclic, aryl, and heteroaryl; each R′ is independently selected from the group consisting of hydrogen, Ci-C6 lower alkyl, Ct-Ce lower alkoxy, hydroxy, amino, alkylamino, dialkylamino, cyano, thiol, heteroaryl, aryl, phenyl, heterocyclic, carbocyclic, and halogen; preferably, R′ is hydrogen, methyl, ethyl, propyl, isopropyl, butyl, methoxy, ethoxy, propoxy, isopropoxy, hydroxyl, amino, fluoro, chloro, or bromo; more preferably, R′ is fluoro, hydrogen, or methyl; n is an integer between 3 and 10,000; x is an integer between 1 and 10; preferably, x is an integer between 2 and 6; y is an integer between 1 and 10; preferably, y is an integer between 2 and 6; z is an integer between 1 and 10; preferably, z is an integer between 2 and 6; and derivatives and salts thereof.

In another embodiment, the bis(acrylate ester) unit in the polymer is chosen from the following group of bis(acrylate ester) units (A′-G′):

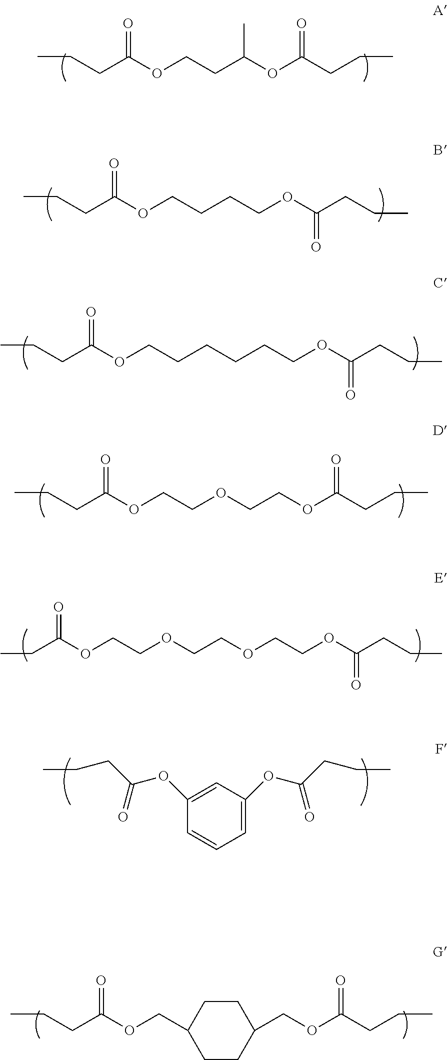

In certain embodiments, the polymer comprises the bis(acrylate ester) G′.

In another embodiment, the bis(acrylate ester) unit in the polymer is chosen from the following group of bis(acrylate ester) units (A-PP):

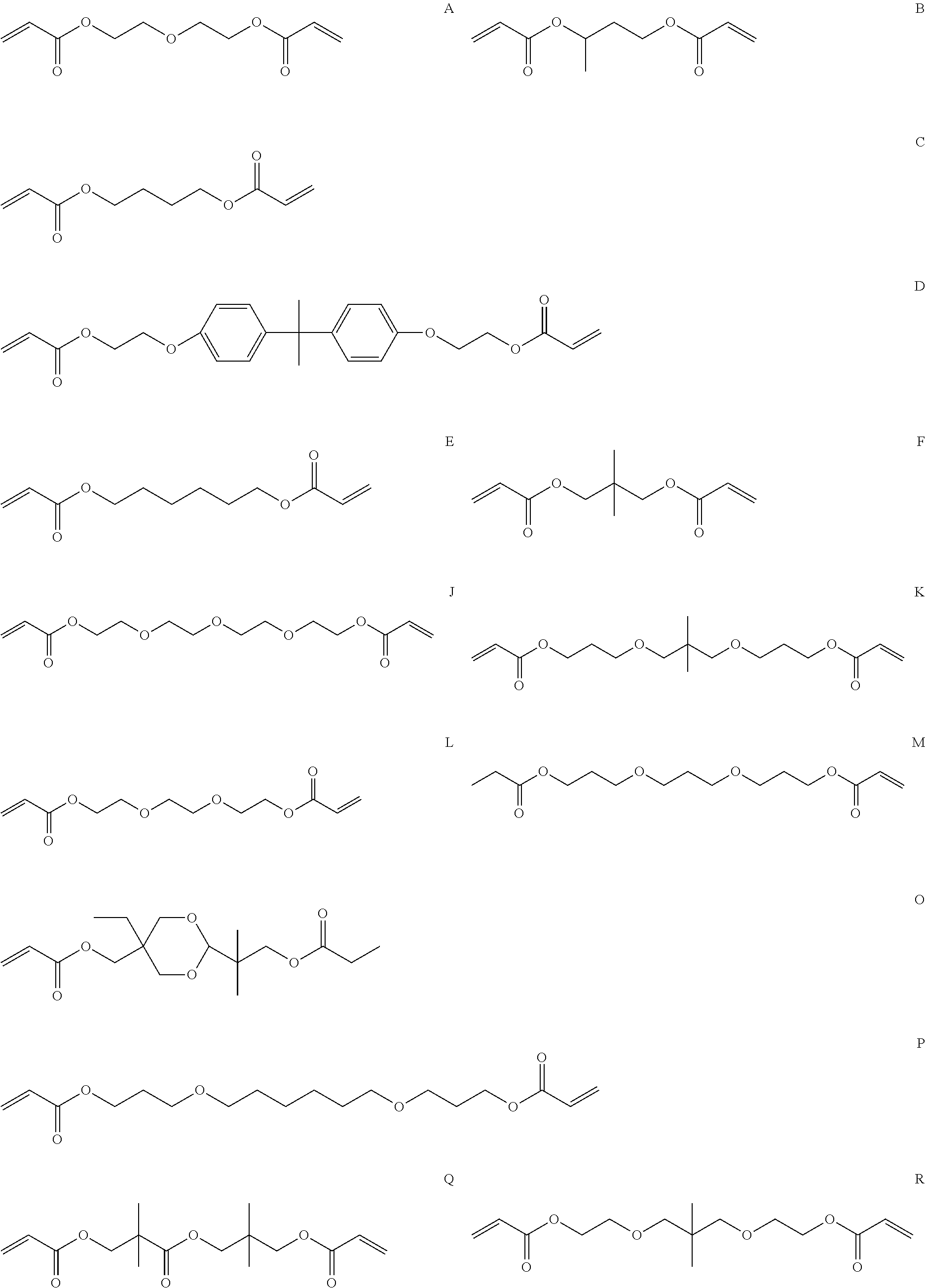

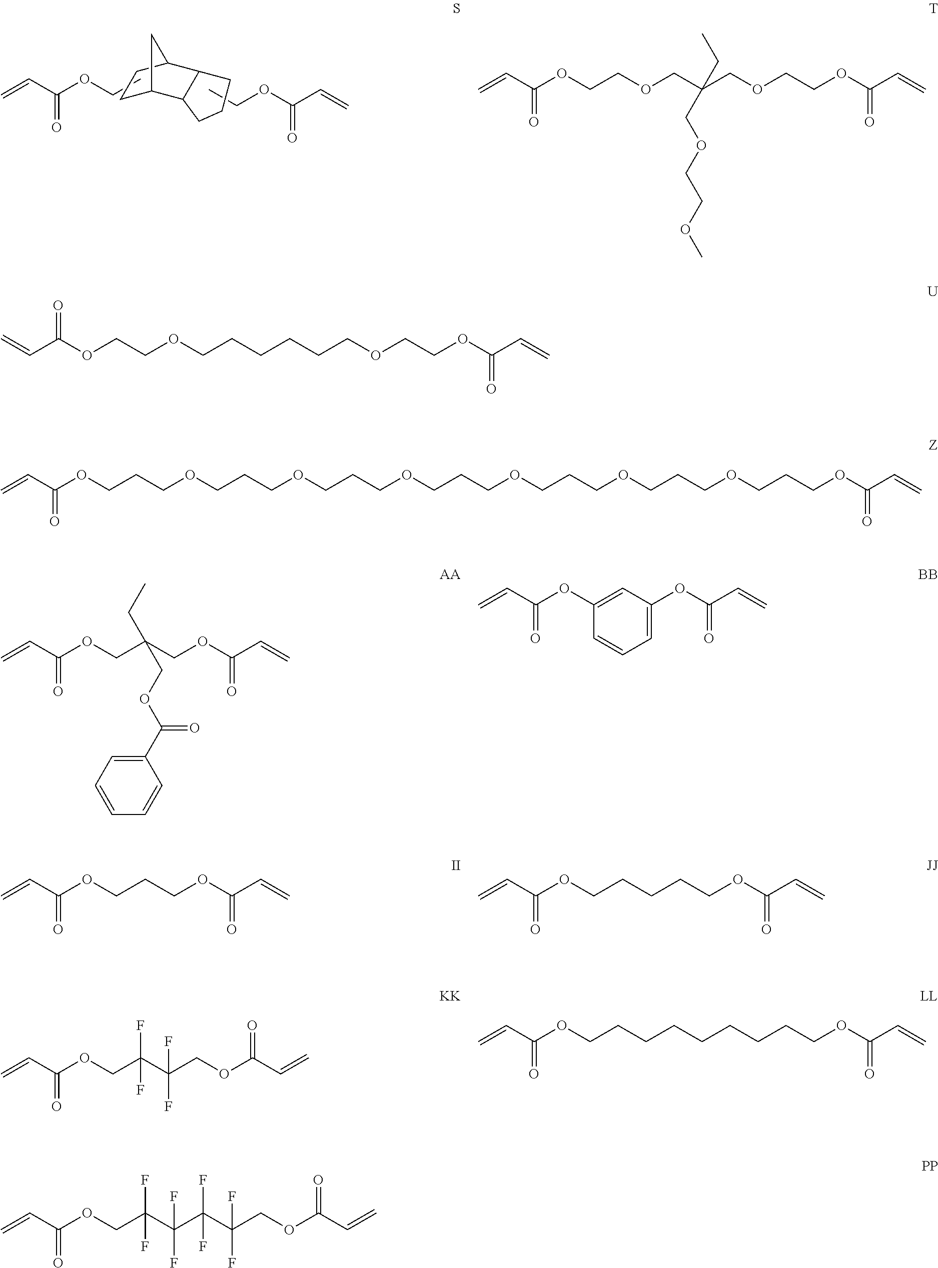

Particularly preferred bis(acrylate esters) in this group include B, C, D, E, F, M, O, U, AA, II, JJ, KK, and LL.

In another embodiment, the amine in the polymer is chosen from the following group of amines (1′-20′):

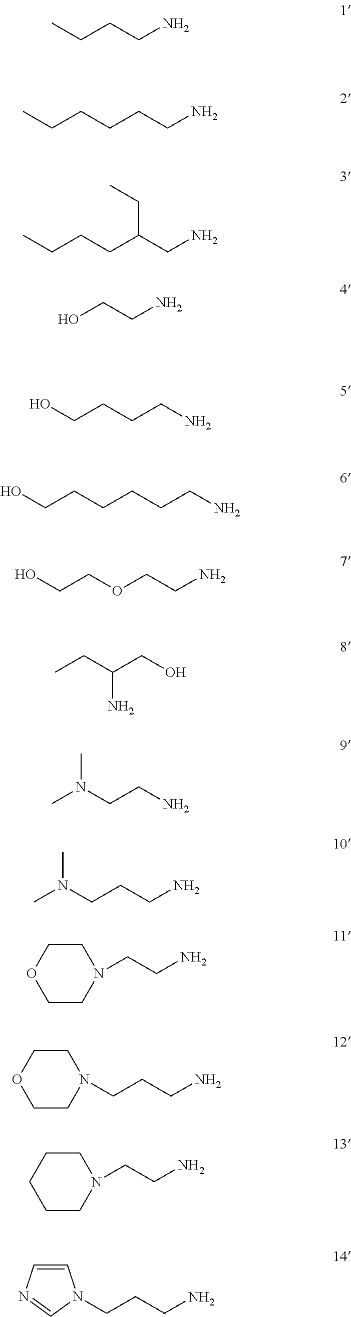

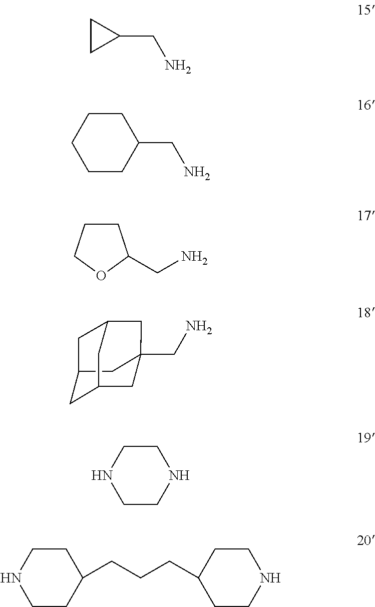

In certain embodiments, the polymer comprises the amine 5′. In other embodiments, the polymer comprises amine 14′.

In another embodiment, the amine in the polymer is chosen from the following group of amines (1-94):

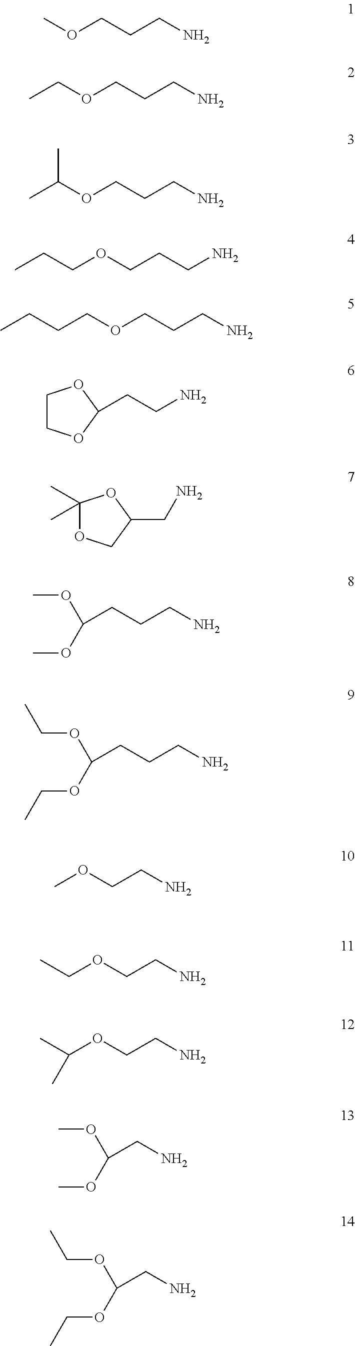

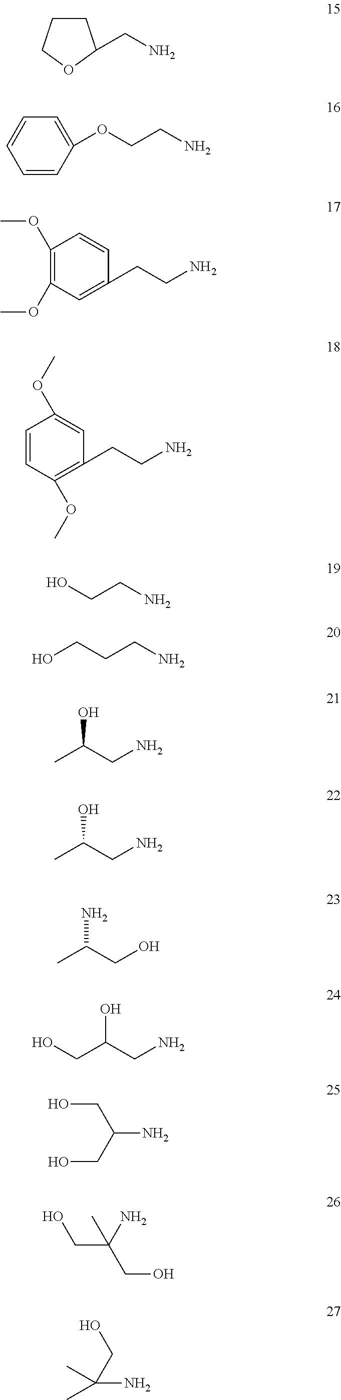

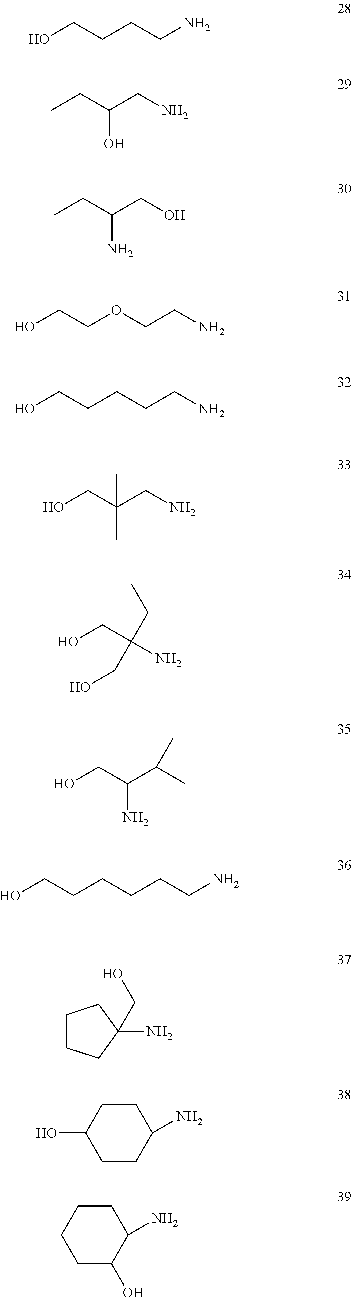

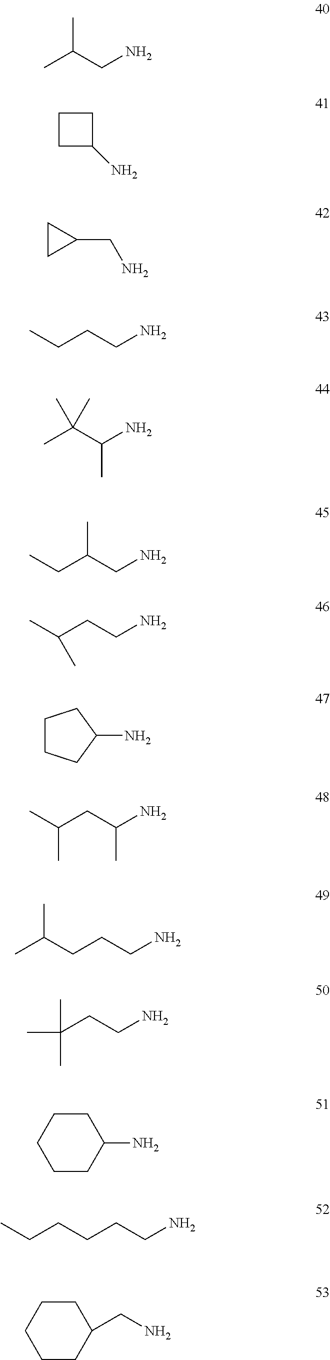

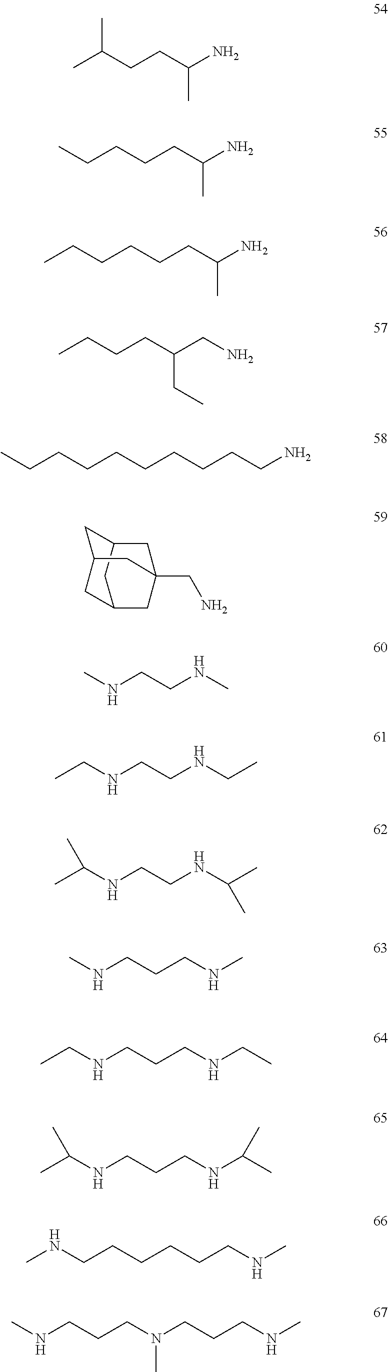

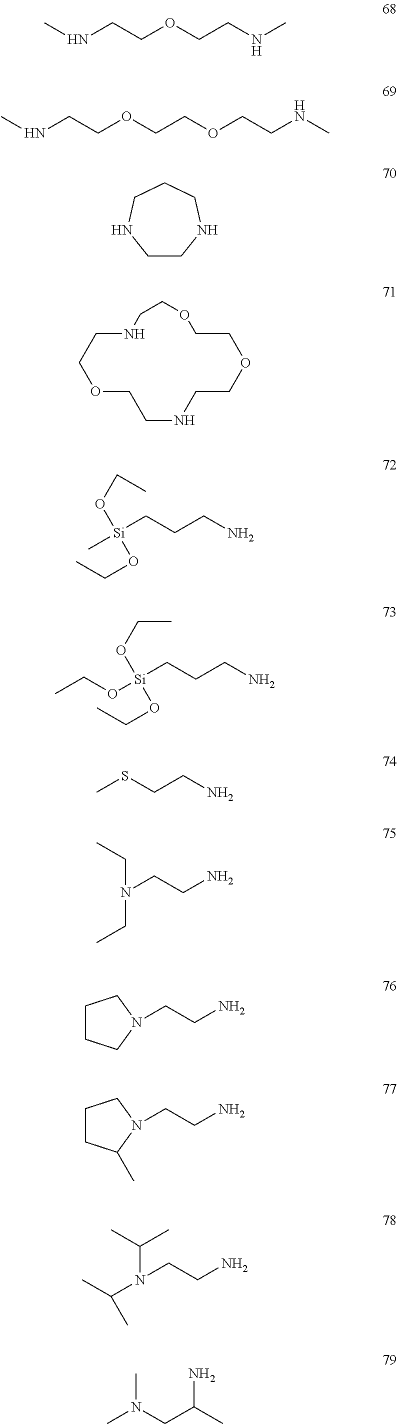

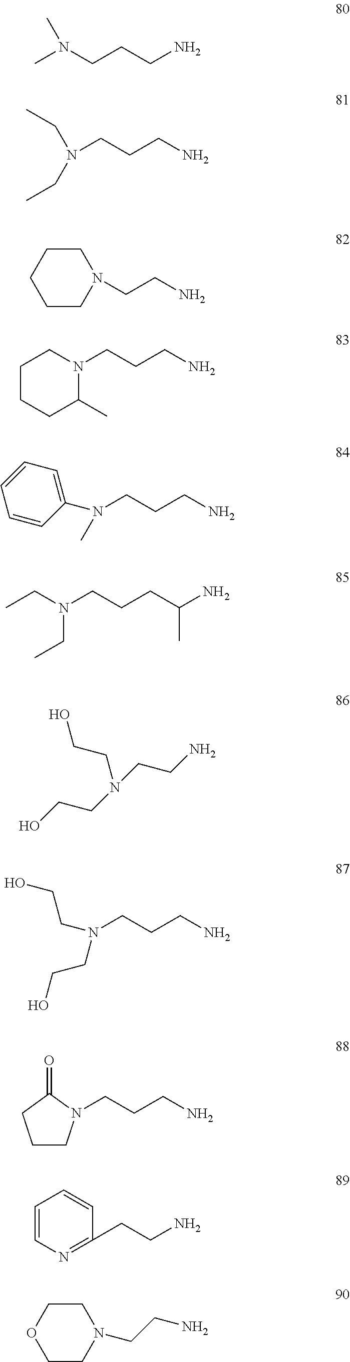

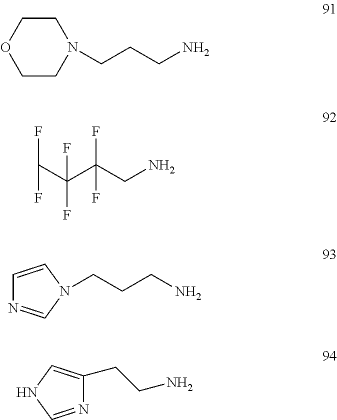

In certain embodiments, the polymers include amines 6, 8, 17, 20, 24, 25, 28, 32, 36, 60, 61, 70, 75, 80, 86, 87, 89, 93, or 94.

Particular examples of the polymers include:

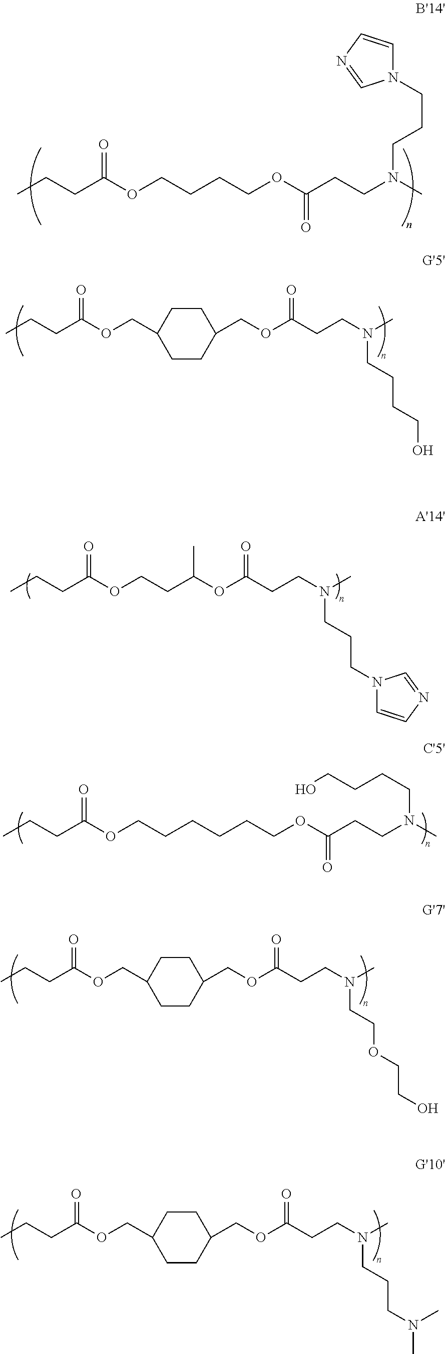

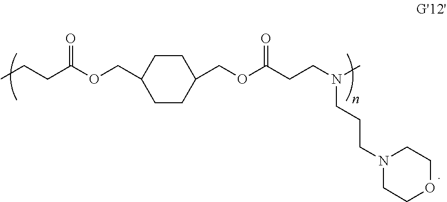

Other particularly useful poly(beta-amino esters) include:

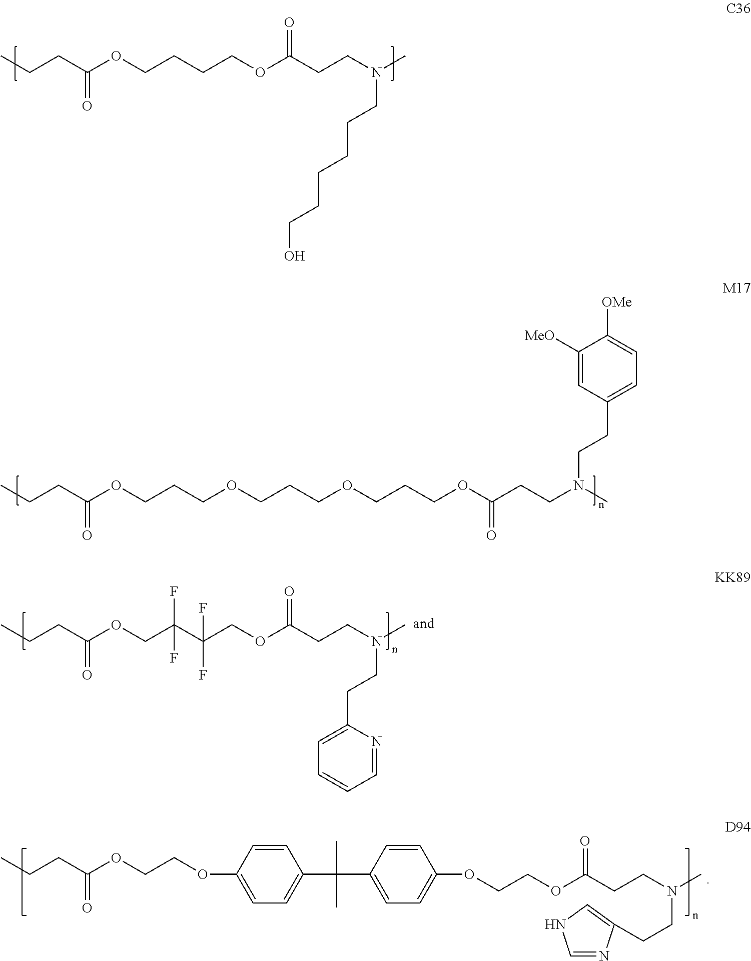

Other particularly useful poly(beta-amino esters) include C86, D60, D61, U94, F32, F28, JJ36, JJ32, LL6, LL8, U28, E28, U36, E36, U32, E32, C94, F94, JJ94, U28, JJ86, C86, U86, E86, C80, E80, JJ80, U80, D24, E24, JJ24, B17, II28, II36, II32, C20, JJ20, E20, C25, U25, D25, D70, D28, D32, D36, D93, U87, D87, C75, U75, 020,028, C94, AA20, AA28, D86, F86, AA36, AA24, AA94,024, AA60, A61, C32, JJ28, C28, JJ20, D94, U32, D24, C36, E28, D36, U94, E24, E32, D28, U36, E80, E36, JJ80, E94, D93, B17, M17, AA61, U93, and C25.

In a particularly preferred embodiment, one or both of the linkers A and B are polyethylene polymers. In another particularly preferred embodiment, one or both of the linkers A and B are polyethylene glycol polymers. Other biocompatible, biodegradable polymers may be used as one or both of the linkers A and B.

In certain preferred embodiments, the polymers are amine-terminated. In other embodiments, the polymers are acrylate-terminated. In large part, the termination unit of the polymer is determined by the ratio of amine versus acrylate in the polymer synthesis reaction. An excess of amine monomer yields an amine-terminated polymer. And an excess of acrylate monomer yields an acrylate-terminated polymer.

In certain embodiments, the average molecular weight of the polymers range from 1,000 g/mol to 50,000 g/mol, preferably from 2,000 g/mol to 40,000 g/mol, more preferably from 5,000 g/mol to 20,000 g/mol, and even more preferably from 10,000 g/mol to 17,000 g/mol. Since the polymers are prepared by a step polymerization, a broad, statistical distribution of chain lengths is typically obtained. In certain embodiments, the distribution of molecular weights in a polymer sample is narrowed by purification and isolation steps known in the art. The molecular weight of the polymer may also be varied by the synthesis of the polymer, for example, as the amount of amine monomers increases, the molecular weight of the resulting polymer decreases. In other embodiments, the polymer mixture may be a blend of polymers of different molecular weights.

In another particularly preferred embodiment, the polymer is a co-polymer wherein one of the repeating units is a poly(beta-amino ester). Other repeating units to be used in the co-polymer include, but are not limited to, polyethylene, poly (glycolide-co-lactide) (PLGA), polyglycolic acid, polymethacrylate, etc.

Synthesis of Polymers

The polymers may be prepared by any method known in the art. Examples are provided, e.g., in WO2004/106411.

Preferably, the polymers are prepared from commercially available starting materials.

In another preferred embodiment, the polymers are prepared from easily and/or inexpensively prepared starting materials.

In a particularly preferred embodiment, the polymer is prepared via the conjugate addition of bis(secondary amines) to bis(acrylate esters). This reaction scheme is shown below:

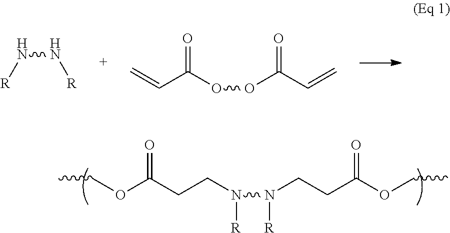

Bis(secondary amine) monomers that are useful in the present method include, but are not limited to, N,N′-dimethylethylenediamine, piperazine, 2-methylpiperazine, 1,2-bis(N-ethylamino)ethylene, and 4,4′-trimethylenedipiperidine.

Diacrylate monomers that are useful in the present invention include, but are not limited to, 1,4-butanediol diacrylate, 1,4-butanediol dimethacrylate, 1,2-ethanediol diacrylate, 1,6-hexanediol diacrylate, 1,5-pentanediol diacrylate, 2,5-hexanediol diacrylate, and 1,3-propanediol diacrylate. Each of the monomers is dissolved in an organic solvent (e.g., THF, CH2C12, MeOH, EtOH, CHC13, hexanes, toluene, benzene, CC14, glyme, diethyl ether, DMSO, DMF, etc.), preferably DMSO. The resulting solutions are combined, and the reaction mixture is heated to yield the desired polymer.

In other embodiments, the reaction is performed without the use of a solvent (i.e, neat) thereby obviating the need for removing the solvent after the synthesis. The reaction mixture is then heated to a temperature ranging from 30° C. to 200 C, preferably 40° C. to 150° C., more preferably 50° C. to 100° C. In a particularly preferred embodiment, the reaction mixture is heated to approximately 40° C., 50° C., 60° C., 70° C., 80° C., or 90° C.

In another particularly preferred embodiment, the reaction mixture is heated to approximately 75° C. In another embodiment, the reaction mixture is heated to approximately 100° C. The polymerization reaction may also be catalyzed. The reaction time may range from hours to days depending on the polymerization reaction and the reaction conditions. As would be appreciated by one of skill in the art, heating the reaction tends to speed up the rate of reaction requiring a shorter reaction time. In certain embodiments, the polymer synthesis is carried out in DMSO at approximately 60° C. for approximately 2 days. In other embodiments, the polymer synthesis is carried out without solvent at 95° C. for 8-16 hours. As would be appreciated by one of skill in this art, the molecular weight of the synthesized polymer may be determined by the reaction conditions (e.g., temperature, starting materials, concentration, catalyst, solvent, time of reaction, etc.) used in the synthesis.

In another particularly preferred embodiment, the polymers are prepared by the conjugate addition of a primary amine to a bis(acrylate ester). The use of primary amines rather than bis(secondary amines) allows for a much wider variety of commercially available starting materials. The reaction scheme using primary amines rather than secondary amines is shown below:

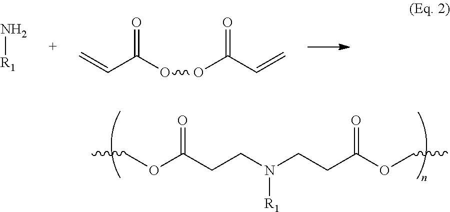

Primary amines useful in this method include, but are not limited to, methylamine, ethylamine, isopropylamine, aniline, substituted anilines, and ethanolamine. The bis(acrylate esters) useful in this method include, but are not limited to, 1,4-butanediol diacrylate, 1,4-butanediol dimethacrylate, 1,2-ethanediol diacrylate, 1,6-hexanediol diacrylate, 1,5-pentanediol diacrylate, 2,5-hexanediol diacrylate, and 1,3-propanediol diacrylate. Each of the monomers is dissolved in an organic solvent (e.g., THF, DMSO, DMF, CH2C12, MeOH, EtOH, CHC13, hexanes, CC14, glyme, diethyl ether, etc.). Organic solvents are preferred due to the susceptibility of polyesters to hydrolysis. Preferably the organic solvent used is relatively non-toxic in living systems. In certain embodiments, DMSO is used as the organic solvent because it is relatively non-toxic and is frequently used as a solvent in cell culture and in storing frozen stocks of cells. Other preferred solvents include those miscible with water such as DMSO, ethanol, and methanol. The resulting solutions of monomers which are preferably at a concentration between 0.01 M and 5 M, between approximately 0.1 M and 2 M, more preferably between 0.5 M and 2 M, and most preferably between 1.3 M and 1.8 M, are combined, and the reaction mixture is heated to yield the desired polymer. In certain embodiments, the reaction is run without solvent. Running the polymerization without solvent may decrease the amount of cyclization products resulting from intramolecular conjugate addition reactions. The polymerization may be run at a temperature ranging from 20° C. to 200° C., preferably from 40° C. to 100° C., more preferably from 50° C. to 75° C., even more preferably from 50° C. to 60° C. In a particularly preferred embodiment, the reaction mixture is maintained at 20° C. In another particularly preferred embodiment, the reaction mixture is heated to approximately 50° C. In some embodiments, the reaction mixture is heated to approximately 56° C. In yet another particularly preferred embodiment, the reaction mixture is heated to approximately 75° C. The reaction mixture may also be cooled to approximately 0° C. The polymerization reaction may also be catalyzed such as with an organometallic catalyst, acid, or base. In another preferred embodiment, one or more types of amine monomers and/or diacrylate monomers may be used in the polymerization reaction. For example, a combination of ethanolamine and ethylamine may be used to prepare a polymer more hydrophilic than one prepared using ethylamine alone, and also more hydrophobic than one prepared using ethanolamine alone.

In preparing the polymers, the monomers in the reaction mixture may be combined in different ratio to effect molecular weight, yield, end-termination, etc. of the resulting polymer. The ratio of amine monomers to diacrylate monomers may range from 1.6 to 0.4, preferably from 1.4 to 0.6, more preferably from 1.2 to 0.8, even more preferably from 1.1 to 0.9. In certain embodiments, the ratio of amine monomers to diacrylate monomers is approximately 1.4, 1.3, 1.2, 1.1, 1.050, 1.025, 1.0, 0.975, 0.950, 0.900, 0.800, and 0.600. For example, combining the monomers at a ratio of 1:1 typically results in higher molecular weight polymers and higher overall yields. Also, providing an excess of amine monomers (i.e., amine-to-acrylate ratio>1) results in amine-terminated chains while providing an excess of acrylate monomer (i.e., amine-to-acrylate ratio<1) results in acrylate-terminated chains. The ratio of amine monomers to acrylate mononers in the polymer synthesis can affect how the polymer chains are terminated, the molecular weight of the polymers produced, the distribution of molecular weights, and the extent of cross-linking.

The synthesized polymer may be purified by any technique known in the art including, but not limited to, precipitation, crystallization, extraction, chromatography, etc. In a particularly preferred embodiment, the polymer is purified through repeated precipitations in organic solvent (e.g., diethyl ether, hexane, etc.). In a particularly preferred embodiment, the polymer is isolated as a hydrochloride, phosphate, acetate, or other salt. Preferably the salt is pharmaceutically acceptable in certain embodiments.

The resulting polymer may also be used as is without further purification and isolation; thereby making it advantageous to use a solvent compatible with the assays to be used in assessing the polymers. For example, the polymers may be prepared in a non-toxic solvent such as DMSO, and the resulting solution of polymer may then be used in cell culture assays involving transfecting a nucleic acid into a cell. As would be appreciated by one of skill in this art, the molecular weight of the synthesized polymer and the extent of cross-linking may be determined by the reaction conditions (e.g., temperature, starting materials, concentration, equivalents of amine, equivalents of acrylate, order of addition, solvent, etc.) used in the synthesis (Odian Principles of Polymerization 3rd Ed., New York: John Wiley & Sons, 1991; Stevens Polymer Chemistry: An Introduction 2nd Ed., New York: Oxford University Press, 1990). The extent of cross-linking of the prepared polymer may be minimized to between 1-20%, preferably between 1-10%, more preferably between 1-5%, and most preferably less than 2%. As would be appreciated by those of skill in this art, amines or bis(acrylate ester) s with nucleophilic groups are more susceptible to cross-linking, and measures may need to be taken to reduce cross-linking such as lowering the temperature or changing the concentration of the starting materials in the reaction mixture. Acrylates and other moieties with unsaturation or halogens are also susceptible to radical polymerization which can lead to cross-linking. The extent of radical polymerization and cross-linking may be reduced by reducing the temperature of the reaction mixture or by other means known in the art.

Selected Complexes

The ability of cationic compounds to interact with negatively-charged moieties (e.g., negatively-charged side groups on amino acids, e.g., of a peptide or protein, e.g., therapeutic agent) or surfaces through electrostatic interactions is well known. The interaction of the polymer with the negatively-charged moiety or surface may at least partially prevent the degradation of moiety, or therapeutic agent containing it. By neutralizing the charge on the negatively-charged moiety, the neutral or slightly-positively-charged complex may also be able to more easily pass through hydrophobic membranes (e.g., cytoplasmic, lysosomal, endosomal, nuclear) of the cell. In a particularly preferred embodiment, the complex is slightly positively charged. In another particularly preferred embodiment, the complex has a positive -potential, more preferably the -potential is between +1 and +30. In certain embodiments, agents such as polyacrylic acid (pAA), poly aspartic acid, polyglutamic acid, or poly-maleic acid may be used to prevent the serum inhibition of the therapeutic agent/polymer complexes in cultured cells in media with serum.

The poly(beta-amino esters) possess tertiary amines in the backbone of the polymer. Although these amines are more hindered, they are available to interact with a therapeutic agent. The therapeutic agent is contacted with the polymers under conditions suitable to form the therapeutic agent/polymer complexes. In certain embodiments, the ratio of nitrogen in the polymer (N) to negative charge in the therapeutic agent is 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 110:1, or 120:1. In certain embodiments, the polymer-to-therapeutic agent (w/w) ratio is 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 110:1, 120:1, 130:1, 140:1, 150:1, or 200:1. The polymer is preferably at least partially protonated so as to form a complex with a negatively charged therapeutic agent. In a preferred embodiment, the therapeutic agent/polymer complexes form nanoparticles that are useful in the delivery of therapeutic agents to cells. In a particularly preferred embodiment, the diameter of the nanoparticles ranges from 50-500 nm, more preferably the diameter of the nanoparticles ranges from 50-200 nm, and most preferably from 90-150 nm. The nanoparticles may be associated with a targeting agent as described below.

In certain embodiments, other agents may be added to the therapeutic agent/poly(beta-amino ester) complexes. In certain embodiments, a co-complexing agent is used. Co-complexing agents usually have a high nitrogen density. Polylysine (PLL) and polyethylenimine (PEI) are two examples of polymeric co-complexing agents. PLL has a molecular weight to nitrogen atom ratio of 65, and PEI has a molecular weight to nitrogen atom ratio of 43. Any polymer with a molecular weight to nitrogen atom ratio in the range of 10-100, preferably 25-75, more preferably 40-70, may be useful as a co-complexing agent. The inclusion of a co-complexing agent in a complex may allow one to reduce the amount of poly(beta-amino ester) in the complex. This becomes particularly important if the poly(beta-amino ester) is cytotoxic at higher concentrations. In the resulting complexes with co-complexing agents, the co-complexing agent to therapeutic agent (w/w) ratio may range from 0 to 2.0, preferably from 0.1 to 1.2, more preferably from 0.1 to 0.6, and even more preferably from 0.1 to 0.4.

Some commercially available cationic compounds include:

| | |
|---|---|
| Ozbiosciences | Lipid Ion Interaction |
| Promega | Cationic Lipids |
| Langer | Lipoid Cationic Lipids |
| Invitrogen | Lipofectamine (Lipid based) |
| Stratagene | Statisfection-Cationic polymer, non-lipid |
| Upstate | silmporter (Cationic Lipid) |
| Roche | Cationic Lipid Formulations |

Drug Delivery Devices

Nanoparticles are polymeric particles in the nanometer size range whereas microparticles are particles in the micrometre size range. Both types of particle can be used as drug delivery devices. The poly(beta-amino esters) may also be used to form drug delivery devices (e.g., microparticles and/or nanoparticles), e.g., to deliver therapeutic agent (e.g., a therapeutic protein, e.g., a protein for use in replacement therapy, e.g., a protein described herein). The polymers may be used to encapsulate therapeutic agents, e.g., therapeutic proteins, e.g., proteins for replacement therapy, e.g., as described herein. The polymers have several properties that make them particularly suitable in the preparation of drug delivery devices. These include 1) the ability of the polymer to complex and “protect” labile agents; 2) the ability to buffer the pH in the endosome; 3) the ability to act as a “proton sponge” and cause endosomolysis; and 4) the ability to neutralize the charge on negatively charged agents.

In a preferred embodiment, the polymers are used to form microparticles and/or nanoparticles containing the therapeutic agent to be delivered. In a particularly preferred embodiment, the diameter of the microparticles ranges from between 500 nm to 50 micrometers, more preferably from 1 micrometer to 20 micrometers, and most preferably from 1 micrometer to 10 micrometers. In another particularly preferred embodiment, the microparticles range from 1-5 micrometers. Nanoparticles can be smaller in size (e.g., between 100 nanometers and 1 micron).

The encapsulating polymer may be combined with other polymers (e.g., PEG, PLGA, poly anhydrides, polyesters, polyamides, polyimides, modified oligosaccharides (Fidia's HA technology), oligosaccharides (HA, CMC, Chitosan, Dextran Derivatives, Alginate, etc.)) to form the microspheres (e.g., microparticles or nanoparticles).

Microparticles and/or nanoparticles formed from biodegradable polymers are attractive for use as delivery devices, e.g., to deliver therapeutic agent (e.g., a therapeutic protein, e.g., a protein for use in replacement therapy, e.g., a protein described herein). For some therapeutic agents (e.g., therapeutic proteins, e.g., proteins for replacement therapy, e.g., as described herein) that require intracellular administration and trafficking to the cytoplasm, there is an increasing demand for new materials that facilitate triggered release in response to environmental stimuli such as pH (Zauner et al. Adv. Drug Del. Rev. 30: 97-113, 1998). Following endocytosis, the pH within cellular endosomal compartments is lowered, and foreign material may be degraded upon fusion with lysosomal vesicles (Kabanov et al. Bioconjugate Chem. 6: 7-20, 1995). New materials that release molecular payloads upon changes in pH within the intracellular range and facilitate escape from hostile intracellular environments could have a fundamental and broad-reaching impact on the administration of hydrolytically- and/or enzymatically-labile drugs (Zauner et al. Adv. Drug Del. Rev. 30: 97-113, 1998; Kabanov et al. Bioconjugate Chem. 6: 7-20, 1995). The fabrication of pH-responsive polymer microspheres that release encapsulated contents quantitatively and essentially instantaneously upon changes in pH within the intracellular range is described in WO2004/106411. In some embodiments, the pH-responsive polymer microspheres are used in the preparation of the therapeutic complexes described herein.

Methods of Preparing Microparticles

The microparticles may be prepared using any method known in this art. These include, but are not limited to, spray drying, single and double emulsion solvent evaporation, solvent extraction, phase separation, simple and complex coacervation, and other methods well known to those of ordinary skill in the art. Nanoparticles can be created by any technique well known in the art. They can be created in the same or similar manner as microparticles. In some embodiments, high-speed mixing or homogenization can be used to reduce the size of the polymer/emulsions to less than 2 microns (see, e.g., WO 97/04747).

Particularly preferred methods of preparing the particles are the double emulsion process and spray drying. The conditions used in preparing the microparticles and/or nanoparticles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the polymer matrix.

Methods developed for making microparticles for delivery of encapsulated agents are described in the literature (for example, see Doubrow, M., Ed., “Microcapsules and Nanoparticles in Medicine and Pharmacy,” CRC Press, Boca Raton, 1992; Mathiowitz and Langer, J Controlled Release 5: 13-22, 1987; Mathiowitz et al. Reactive Polymers 6: 275-283, 1987; Mathiowitz et al. J. Appl. Polymer Sci. 35: 755-774, 1988).

If the particles prepared by any of the above methods have a size range outside of the desired range, the particles can be sized, for example, using a sieve.

Targeting Agents

The micro- and nanoparticles may be modified to include targeting agents since it is often desirable to target a particular cell, collection of cells, or tissue.

A variety of targeting agents that direct pharmaceutical compositions to particular cells are known in the art (see, for example, Cotten et al. Methods Enzym. 217: 618, 1993). The targeting agents may be included throughout the particle or may be only on the surface. The targeting agent may be a protein, peptide, carbohydrate, glycoprotein, lipid, small molecule, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp 120 envelope protein of the human immunodeficiency virus (HIV), carbohydrates, receptor ligands, sialic acid, etc. If the targeting agent is included throughout the particle, the targeting agent may be included in the mixture that is used to form the particles. If the targeting agent is only on the surface, the targeting agent may be associated with (i.e., by covalent, hydrophobic, hydrogen boding, van der Waals, or other interactions) the formed particles using standard chemical techniques. The targeting strategy may also include several different targeting moieties (Journal of Controlled Release 120 (2007) 242-249)

Targeting strategy may also involve the reductive cleavage of a sensitive bond between the protein and the polymer (e.g., strategic disulfide insertions) (Journal of Controlled Release 120 (2007) 250-258.

Pharmaceutical Compositions

Once the therapeutic compositions, e.g., microparticles, or nanoparticles (e.g., containing a polymer described herein complexed with a therapeutic agent (e.g., a therapeutic protein, e.g., a protein for use in replacement therapy, e.g., a protein described herein)) have been prepared, they may be combined with one or more pharmaceutical excipients to form a pharmaceutical composition that is suitable to administer to animals including humans. As would be appreciated by one of skill in this art, the excipients may be chosen based on the route of administration as described below, the agent being delivered, time course of delivery of the agent, etc.

Pharmaceutical compositions of the present invention and for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., microparticles, nanoparticles, polynucleotide/polymer complexes), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In a particularly preferred embodiment, the particles are suspended in a carrier fluid comprising 1% (w/v) sodium carboxymethyl cellulose and 0.1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin,) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient (s) only, or preferentially, in a certain part of the intestinal tract, optionally, in a delayed manner.

Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Dosage forms for topical or transdermal administration of an pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the particles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the particles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the microparticles or nanoparticles in a proper medium. Absorption enhancers can also be used to increase the flux of the compound across the skin. The rate can be controlled by either providing a rate controlling membrane or by dispersing the particles in a polymer matrix or gel.

These and other aspects of the present invention will be further appreciated upon consideration of the following Examples, which are intended to illustrate certain particular embodiments of the invention but are not intended to limit its scope, as defined by the claims.

### EXAMPLES

For use in the preparation of therapeutic complexes containing poly(beta-amino esters) polymers and a therapeutic agent described herein, examples of how to prepare the polymers are as follows.

Briefly, three poly(beta-amino ester) polymers were synthesized via the conjugate addition of N,N′-dimethylethylenediamine, piperazine, and 4,4′-trimethylenedipiperidine to 1,4-butanediol diacrylate. The amines in the bis (secondary amine) monomers actively participate in bond-forming processes during polymerization, obviating the need for amine protection/deprotection processes which characterize the synthesis of other poly(amino esters). Accordingly, this approach enables the generation of a variety of structurally diverse polyesters containing tertiary amines in their backbones in a single step from commercially available staring materials. The methods are detailed in Examples 1, 3, 4, 5, and 6 of WO2004/106411.

Formation of Protein/Polymer Complexes: Complexes that contain a therapeutic protein described herein and a polymer described herein are prepared. Protein/polymer complexes are formed by adding 50 μL of a protein solution (to a gently vortexing solution of the hydrochloride salt of polymers (50 μL in 25 mM MES, pH=6.0, concentrations adjusted to yield desired protein/polymer weight ratios). The samples are incubated at room temperature for 30 minutes, after which an aliquot is run on a non-denaturing gel (polyacrylamide gel electrophoresis (PAGE)). The percent acrylamide of the gel is determined based on the size of the protein being complexed. The protein is visualized by Coomassie brilliant blue staining. A control aliquot of uncomplexed protein is also run for size comparisons.

A number of embodiments of the invention have been described. Nevertheless, it will be understood that various modifications may be made without departing from the spirit and scope of the invention. Accordingly, other embodiments are within the scope of the following claims.

What is claimed is:

1. A therapeutic composition comprising a polymer that comprises a poly(beta-amino ester) and a therapeutic agent, wherein the therapeutic agent comprises a protein.

2. The therapeutic composition of claim 1, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

3. A therapeutic composition comprising a therapeutic protein and a compound of the formula: 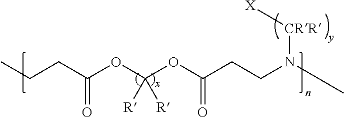
wherein
X is methyl, OR or NR2;
R is selected from the group consisting of hydrogen, alkyl, alkenyl, alkynyl, cyclic, heterocyclic, aryl, and heteroaryl; each R′ is independently selected from the group consisting of hydrogen, C1-C6 lower alkyl, C1-C6 lower alkoxy, hydroxy, amino, alkylamino, dialkylamino, cyano, thiol, heteroaryl, aryl, phenyl, heterocyclic, carbocyclic, and halogen; n is an integer between 3 and 10,000; x is an integer between 1 and 10; y is an integer between 1 and 10; and derivatives and salts thereof.

4. The therapeutic composition of claim 3, wherein the therapeutic protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

5. A pharmaceutical composition comprising the therapeutic composition of claim 1.

6. A pharmaceutical composition comprising nanoparticles that comprise the therapeutic composition of claim 1.

7. A pharmaceutical composition comprising microparticles that comprise the therapeutic composition of claim 1 encapsulated in a matrix.

8. The pharmaceutical composition of claim 7, wherein the microparticles have a mean diameter of 1-10 micrometers.

9. The pharmaceutical composition of claim 7, wherein the microparticles have a mean diameter of less than 5 micrometers.

10. The pharmaceutical composition of claim 7, wherein the microparticles have a mean diameter of less than 1 micrometer.

11. A method of making a therapeutic composition, the method comprising:
providing a poly(beta-amino ester) described herein;
providing a therapeutic agent described herein; and
combining the poly(beta-amino ester) and the therapeutic agent, thereby making a therapeutic composition.

12. The method of claim 11, wherein the therapeutic agent is a therapeutic protein that comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

13. A method of treating an individual, the method comprising:
administering the therapeutic composition of claim 1 to an individual in need of such treatment.

14. The method of claim 13, wherein the individual is in need of replacement therapy.

15. The method of claim 13, wherein the therapeutic composition comprises a therapeutic agent that comprises a therapeutic protein that comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

16. A method of preparing microparticles, the method comprising:
contacting a therapeutic agent described herein with a poly(beta-amino ester) described herein in the presence of a solvent to form a mixture; and
spray drying the mixture, thereby preparing microparticles.
